(19) United States
(12) Patent Application Publication
(10) Pub. No.: US 2023/0389852 A1
(43) Pub. Date: Dec. 7, 2023

(54) METHODS, DEVICES AND SYSTEMS THAT USE ONE OR MORE TRANSDUCERS TO HEAT NERVES TO EVOKE NEURAL RESPONSE WITHOUT DENERVATING NERVES, AS WELL AS TO DENERVATE NERVES

(71) Applicant: Otsuka Medical Devices Co., Ltd., Tokyo (JP)

(72) Inventor: Liang Zhai, Belmont, CA (US)

(21) Appl. No.: 18/327,280

(22) Filed: Jun. 1, 2023

Related U.S. Application Data
(60) Provisional application No. 63/365,771, filed on Jun. 2, 2022.

(51) Int. Cl. A61B 5/388; A61B 18/14; A61B 18/18; A61N 7/02

(52) U.S. Cl. CPC A61B 5/388; A61B 18/1492; A61B 18/1815; A61N 7/022; A61B 2018/1861; A61B 2018/00702

(57) ABSTRACT

Certain methods and systems described herein use a transducer used to produce ablation energy (aka denervation energy) to also evoke a neural response before and/or after denervation energy is delivered. Rather than the neural response being invoked in response to electrical stimulus, it is invoked in response to the transducer (e.g., RF, microwave, or ultrasound transducer) being used to sufficiently heat the nerves in tissue surrounding a biological lumen to an extent that a neural response is evoked without denervating the nerves. That is, the transducer used for performing ablation (aka denervation) is also used to evoke a neural response prior to (and/or after) ablation is performed, by using the transducer to sufficiently heat the nerves in the tissue surrounding the biological lumen to an extent that a neural response is evoked without denervating the nerves. This can reduce the catheter production complexity and costs. Other embodiments are also disclosed.

### PRIORITY CLAIM

The present application claims priority to U.S. Provisional Patent Application No. 63/365,771, filed Jun. 2, 2022, which is incorporated herein by reference in its entirety to provide continuity of disclosure.

### FIELD

Embodiments of the present technology generally relate to methods, devices and system for heating nerves to evoke a neural response, for the purpose of sensing a baseline neural response prior to ablation and/or to sensing a post-ablation neural response.

### BACKGROUND

The human body's nervous system includes both the somatic nervous system that provides sense of the environment (vision, skin sensation, etc.) and regulation of the skeletal muscles, and is largely under voluntary control, and the autonomic nervous system, which serves mainly to regulate the activity of the internal organs and adapt them to the body's current needs, and which is largely not under voluntary control. The autonomic nervous system involves both afferent or sensory nerve fibers that can mechanically and chemically sense the state of an organ, and efferent fibers that convey the central nervous system's response (sometimes called a reflex arc) to the sensed state information. In some cases, the somatic nervous system is also influenced, such as to cause vomiting or coughing in response to a sensed condition.

Regulation of the human body's organs can therefore be somewhat characterized and controlled by monitoring and affecting the nerve reflex arc that causes organ activity. For example, the renal nerves leading to a kidney can often cause a greater reflexive reaction than desired, contributing significantly to hypertension. Measurement of the nerve activity near the kidney, and subsequent ablation of renal nerves can therefore be used to control the nervous system's overstimulation of the kidney, improving operation of the kidney and the body as a whole.

Because proper operation of the nervous system is therefore an important part of proper organ function, it is desired to be able to monitor and change nervous system function in the human body to characterize and correct nervous system regulation of internal human organs.

New medical therapies have been practiced whereby a probe such as a needle, catheter, wire, etc. is inserted into the body to a specified anatomical location and destructive means are conveyed to nerves by means of the probe to irreversibly damage tissue in the nearby regions. The objective is to modulate (e.g., abolish) nerve function in the specified anatomic location. The result is that abnormally functioning physiological processes can be terminated or modulated back into a normal range. Unfortunately, such medical therapies are not always successful because there is no means to assess that the nervous activity has been successfully abolished. An alternative objective can be to increase a physiologic process or modulate it to an abnormal range.

An example is renal nerve ablation to relieve hypertension. Various studies have confirmed the relationship of renal nerve activity with blood pressure regulation. In various renal ablation procedures, a catheter is introduced into a hypertensive patient's arterial vascular system and advanced into the renal artery. Renal nerves are located in the arterial wall and/or in regions adjacent to the artery. Destructive means are delivered proximate to the renal artery wall to an extent intended to cause destruction of nerve activity. Destructive means include energy such as radio frequency (RF), microwave, cryotherapy, ultrasound, laser or chemical agents. The objective is to reduce or abolish the renal nerve activity. Such nerve activity is an important factor in the creation and/or maintenance of hypertension and abolishment (or reduction) of the nerve activity reduces blood pressure and/or medication burden.

Unfortunately not all patients respond to this therapy. Renal nerve ablation procedures are sometimes ineffective, potentially due to a poor probe/tissue interface. Accordingly, insufficient quantities of destructive means are delivered to the nerve fibers transmitting along the renal artery. One reason is that the delivery of destructive means to the arterial wall does not have a feedback mechanism to assess the destruction of the nerve activity. As a consequence an insufficient quantity of destructive means is delivered and nervous activity is not sufficiently reduced or abolished. Clinicians, therefore, require a means of improving the probe/tissue interface or better targeting of nerves, and a technology to monitor the integrity of the nerve fibers passing through the arterial wall in order to confirm destruction of nerve activity prior to terminating therapy. Current technology for the destruction of nerve activity does not provide practitioners with a feedback mechanism to detect when the desired nervous activity destruction is accomplished. Nerve destructive means are applied empirically without knowledge that the desired effect has been achieved.

It is known that ablation of the renal nerves, with sufficient energy, is able to effect a reduction in both systolic and diastolic blood pressure. Current methods are said to be, from an engineering perspective, open loop; i.e., the methods used to effect renal denervation do not employ any way of measuring, in an acute clinical setting, the results of applied ablation energies. It is only after application of such energies and a period of time (1-12 months) that the effects of the procedure are known.

The two major components of the autonomic nervous system (ANS) are the sympathetic and the parasympathetic nerves. The standard means for monitoring autonomic nerve activity in situations such as described is to insert very small electrodes into the nerve body or adjacent to it. The nerve activity creates an electrical signal in the electrodes which is communicated to a monitoring means such that a clinician can assess nerve activity. This practice is called microneurography and its practical application is by inserting the electrodes transcutaneously to the desired anatomical location. This is not possible in the case of the ablation of many autonomic nerves proximate to arteries, such as the renal artery, because the arteries and nerves are located within the abdomen and cannot be accessed transcutaneously with any reliability. Thus, the autonomic nerve activity cannot be assessed in a practical or efficacious manner.

The autonomic nervous system is responsible for regulating the physiological processes of circulation, digestion, metabolism, hormonal function, immune function, reproduction, and respiration, among others. The sympathetic nerves and parasympathetic nerves most often accompany the blood vessels supplying the body organs which they regulate. Examples of such include but are not limited to the following: (1) Nerves regulating liver function accompany the hepatic artery and the portal vein; (2) Nerves regulating the stomach accompany the gastroduodenal, the right gastroepiploic artery, and the left gastric artery; (3) Nerves regulating the spleen accompany the splenic artery; (4) Nerves from the superior mesenteric plexus accompany the superior mesenteric artery, where both the artery and the nerves branch to the pancreas, small intestine, and large intestine; (5) Nerves of the inferior mesenteric plexus accompany the inferior mesenteric artery and branch with the artery to supply the large intestine, the colon, and the rectum; (6) Nerves accompanying the pulmonary artery that regulated the lungs and/or cardiac function; and (7) Greater splanchnic nerves regulating venous pooling.

Deficiencies in the use of existing therapeutic protocols in denervation of autonomic nerves proximate arteries include: 1) The inability to determine the appropriate lesion sites along the artery that correspond to the location of nerves; 2) The inability to verify that the destructive devices are appropriately positioned adjacent to the arterial wall, normalizing the tissue/device interface and enabling energy transfer through the vessel wall; and 3) The inability to provide feedback to the clinician intraoperatively to describe lesion completeness or the integrity of the affected nerve fibers. As a consequence, autonomic nerve ablation procedures are typically performed in a ‘blind’ fashion; the clinician performing the procedure does not know where the nerves are located; and further, whether the nerves have truly been ablated. Instead, surrogates such as calf muscle sympathetic activity (MSNA) or catecholamine spillover into the circulating blood have been used to attempt to evaluate the reduction in organ specific autonomic activity such as renal nerve activity. It is entirely likely that this deficiency could at least partly be responsible for the current variability in clinical responses coming from clinical trials. Therefore, a system designed to indicate with precision, and in real time, whether ablation was successful is desirable.

### SUMMARY

Certain embodiments of the present technology described herein related to methods for use with a catheter including one or more electrodes and also including a transducer that can be controlled to emit a selective amount of energy, wherein the methods are for use while at least a distal portion of the catheter is inserted into a biological lumen that is surrounded by tissue including nerves that are to be denervated using the transducer of the catheter. In certain embodiments, such a method includes (a) energizing the transducer of the catheter to emit a first instance of a first amount of energy that is sufficient to heat the nerves in the tissue surrounding the biological lumen to an extent that a neural response is evoked without denervating the nerves. The method also includes (b) using at least one of the one or more electrodes of the catheter to sense the neural response that is evoked by energizing the transducer to emit the first instance of the first amount of energy, and storing first information about the sensed neural response that is evoked by energizing the transducer to emit the first instance of the first amount of energy. The method further includes (c) energizing the transducer of the catheter to emit a first instance of a second amount of energy that is sufficient to denervate at least some the nerves in the tissue surrounding the biological lumen. Additionally, the method includes (d) after energizing the transducer of the catheter to emit the first instance of the second amount of energy, energizing the transducer of the catheter to emit a second instance of the first amount of energy that is sufficient to heat the nerves in the tissue surrounding the biological lumen to an extent that a neural response is evoked without denervating the nerves. The method additionally includes (e) using at least one of the one or more electrodes of the catheter to sense the neural response that is evoked by energizing the transducer to emit the second instance of the first amount of energy, and storing second information about the sensed neural response that is evoked by energizing the transducer to emit the second instance of the first amount of energy. The method further includes (f) comparing the second information to the first information and using results of the comparing to determine to what extent the nerves in the tissue surrounding the biological lumen were sufficiently denervated in response to the emission of the first instance of the second amount of energy.

In accordance with certain embodiments, such a method further includes (g) in response to determining that the nerves in the tissue surrounding the biological lumen were not sufficiently denervated in response to the emission of the first instance of the second amount of energy, energizing the transducer of the catheter to emit a second instance of the second amount of energy. Alternative, such a method further includes (g) in response to determining that the nerves in the tissue surrounding the biological lumen were not sufficiently denervated in response to the emission of the first instance of the second amount of energy, energizing the transducer of the catheter to emit a third amount of energy that is greater than the second amount of energy.

In accordance with certain embodiments, the first amount of energy emitted from the transducer, that is sufficient to heat the nerves in the tissue surrounding the biological lumen to the extent that a neural response is evoked without denervating the nerves, is provided by causing the transducer to emit energy within a specified frequency range for a first duration and having a first power level that are collectively sufficient to heat the tissue surrounding the biological lumen to a temperature of at least 38 degrees Celsius, but not to exceed 52 degrees Celsius. The second amount of energy emitted from the transducer, that is sufficient to denervate at least some the nerves in the tissue surrounding the biological, is provided by causing the transducer to emit energy within the same specified frequency range for a second duration and having a second power level that are collectively sufficient to heat the tissue surrounding the biological lumen to a temperature of at least 43 degrees Celsius, but not to exceed 100 degrees Celsius. The second duration is greater than the first duration and/or the second power level is greater than the first power level.

In accordance with certain embodiments, the second duration is greater than the first duration, and the second power level is greater than the first power level. Alternatively, the second duration is greater than the first duration, and the second power level is the same as the first power level. Alternatively, the second duration is the same as the first duration, and the second power level is greater than the first power level.

In accordance with certain embodiments, the transducer of the catheter comprises an ultrasound transducer, and the specified frequency range comprises 5 MHz to 20 MHz. Alternatively, the transducer of the catheter comprises a radio frequency (RF) transducer, and the specified frequency range comprises 300 kHz to 600 kHz. Alternatively, the transducer of the catheter comprises a microwave transducer, and the specified frequency range comprises 300 MHz to 30 GHz.

In accordance with certain embodiments, the (b) using at least one of the one or more electrodes of the catheter to sense the neural response that is evoked by energizing the transducer to emit the first instance of the first amount of energy, comprises one of the following: using a pair of electrodes of the catheter to sense the neural response; using an electrode of the catheter and an electrode of a guidewire to sense the neural response; using an electrode of the catheter and an electrode of an introducer sheath to sense the neural response; or using an electrode of the catheter and an external skin electrode to sense the neural response.

In accordance with certain embodiments, the (a) energizing the transducer of the catheter to emit the first instance of the first amount of energy, and the (b) using the at least one of the one or more electrodes of the catheter to sense the neural response that is evoked by energizing the transducer to emit the first instance of the first amount of energy, are performed contemporaneously. Similarly, the (d) energizing the transducer of the catheter to emit the second instance of the first amount of energy, and the (e) using the at least one of the one or more electrodes of the catheter to sense the neural response that is evoked by energizing the transducer to emit the second instance of the first amount of energy, are performed contemporaneously. In certain such embodiments, the method further comprises using a lowpass filter or a bandpass filter to distinguish the sensed neural responses to the energy emitted by the transducer from the energy emitted by the transducer.

In accordance with certain embodiments, after the (a) energizing the transducer of the catheter to emit the first instance of the first amount of energy, after the (b) using the at least one of the one or more electrodes of the catheter to sense the neural response that is evoked by energizing the transducer to emit the first instance of the first amount of energy, and prior to the (d) energizing the transducer of the catheter to emit the second instance of the first amount of energy, the method further comprises determining one or more characteristics of the sensed neural response of the nerves within the tissue surrounding the biological lumen, the one or more characteristics indicative of one or more of a size, type, function or health of the nerves and/or indicative of proximity of the nerves relative to the at least one of the one or more electrodes of the catheter. The method also includes selecting, based on the one or more characteristics of the sensed neural response, one or more denervation parameters to be used for the (c) energizing the transducer of the catheter to emit the first instance of the second amount of energy that is sufficient to denervate at least some the nerves in the tissue surrounding the biological lumen.

Embodiments of the present technology are also directed to systems that include a catheter, memory, an excitation source, a sensing subsystem, and a controller. The catheter includes one or more electrodes and also includes a transducer that can be controlled to emit a selective amount of energy, wherein the catheter is configured such that at least a distal portion of the catheter is insertable into a biological lumen that is surrounded by tissue including nerves that are to be denervated using the transducer of the catheter. The excitation source is configured to selectively provide energy to the transducer of the catheter. The sensing subsystem is electrically coupled to at least one of the one or more sensing electrodes of the catheter. The controller is communicatively coupled to the excitation source, the sensing subsystem, and the memory. In certain embodiments, the controller is configured to cause the excitation source to energize the transducer of the catheter to emit a first instance of a first amount of energy that is sufficient to heat the nerves in the tissue surrounding the biological lumen to an extent that a neural response is evoked without denervating the nerves. The controller is also configured to cause the sensing subsystem to sense, using at least one of the one or more electrodes of the catheter, the neural response that is evoked by energizing the transducer to emit the first instance of the first amount of energy, and store in the memory first information about the sensed neural response that is evoked by energizing the transducer to emit the first instance of the first amount of energy. Additionally, the controller is configured to cause the excitation source to energize the transducer of the catheter to emit a first instance of a second amount of energy that is sufficient to denervate at least some the nerves in the tissue surrounding the biological lumen. The controller is also configured to cause the excitation source to energize the transducer of the catheter to emit a second instance of the first amount of energy, after the first instance of the second amount of energy has been emitted. The controller is further configured to cause the sensing subsystem to sense, using at least one of the one or more electrodes of the catheter, the neural response that is evoked by energizing the transducer to emit the second instance of the first amount of energy, and store in the memory second information about the sensed neural response that is evoked by energizing the transducer to emit the second instance of the first amount of energy. Additionally, the controller is configured to compare the second information to the first information and use results of the comparison to determine to what extent the nerves in the tissue surrounding the biological lumen were sufficiently denervated in response to the emission of the first instance of the second amount of energy.

In accordance with certain embodiments, the controller is further configured to cause the excitation source to energize the transducer of the catheter to emit a second instance of the second amount of energy, in response to the controller determining that the nerves in the tissue surrounding the biological lumen were not sufficiently denervated in response to the emission of the first instance of the second amount of energy.

In accordance with certain embodiments, the controller is further configured to cause the excitation source to energize the transducer of the catheter to emit a third amount of energy that is greater than the second amount of energy, in response to the controller determining that the nerves in the tissue surrounding the biological lumen were not sufficiently denervated in response to the emission of the first instance of the second amount of energy.

In accordance with certain embodiments, the first amount of energy emitted from the transducer, that is sufficient to heat the nerves in the tissue surrounding the biological lumen to the extent that a neural response is evoked without denervating the nerves, is provided to the transducer to cause emitting of energy within a specified frequency range for a first duration and having a first power level that are collectively sufficient to heat the tissue surrounding the biological lumen to a temperature of at least 38 degrees Celsius, but not to exceed 52 degrees Celsius. The second amount of energy emitted from the transducer, that is sufficient to denervate at least some the nerves in the tissue surrounding the biological, is provided to the transducer to cause emitting of energy within the same specified frequency range for a second duration and having a second power level that are collectively sufficient to heat the tissue surrounding the biological lumen to a temperature of at least 43 degrees Celsius, but not to exceed 100 degrees Celsius. The second duration is greater than the first duration and/or the second power level is greater than the first power level.

In accordance with certain embodiments, the transducer of the catheter comprises an ultrasound transducer, and the specified frequency range comprises 5 MHz to 20 MHz. Alternatively, the transducer of the catheter comprises a radio frequency (RF) transducer, and the specified frequency range comprises 300 kHz to 600 kHz. Alternatively, the transducer of the catheter comprises a microwave transducer, and the specified frequency range comprises 300 MHz to 30 GHz.

In accordance with certain embodiments, the controller is configured to cause the sensing subsystem to sense the neural response that is evoked by energizing the transducer to emit the first instance of the first amount of energy, contemporaneously with controlling the excitation source to energize the transducer of the catheter to emit the first instance of the first amount of energy. The controller is also configured to sense the neural response that is evoked by energizing the transducer to emit the second instance of the first amount of energy, contemporaneously with controlling the excitation source to energize the transducer of the catheter to emit the second instance of the first amount of energy. In certain such embodiments, the system further comprises a lowpass filter or a bandpass filter configured to distinguish the sensed neural responses to the energy emitted by the transducer from the energy emitted by the transducer.

In accordance with certain embodiments, the controller is further configured to determine one or more characteristics of the sensed neural response that is evoked by energizing the transducer to emit the first instance of the first amount of energy, the one or more characteristics indicative of one or more of a size, type, function or health of the nerves and/or indicative of proximity of the nerves relative to the at least one of the one or more electrodes of the catheter. Additionally, the controller is configured to select, based on the one or more characteristics of the sensed neural response, one or more denervation parameters to be used for energizing the transducer of the catheter to emit the first instance of the second amount of energy that is sufficient to denervate at least some the nerves in the tissue surrounding the biological lumen.

A method according to certain embodiments of the present technology includes energizing the transducer of the catheter to emit a first amount of energy that is sufficient to heat the nerves in the tissue surrounding the biological lumen to an extent that a neural response is evoked without denervating the nerves. The method also includes using at least one of the one or more electrodes of the catheter to sense the neural response that is evoked by energizing the transducer to emit the first amount of energy. The method further includes determining one or more characteristics of the sensed neural response of the nerves within the tissue surrounding the biological lumen, the one or more characteristics indicative of one or more of a size, type, function or health of the nerves and/or indicative of proximity of the nerves relative to the at least one of the one or more electrodes of the catheter. Additionally, the method includes selecting, based on the one or more characteristics of the sensed neural response, one or more denervation parameters to be used for energizing the transducer of the catheter to emit a second amount of energy that is sufficient to denervate at least some the nerves in the tissue surrounding the biological lumen. Further, the method includes using the selected one or more denervation parameters for energizing the transducer of the catheter to emit the second amount of energy that is sufficient to denervate at least some the nerves in the tissue surrounding the biological lumen.

A system according to certain embodiments of the present technology includes a catheter, memory, an excitation source, a sensing subsystem, and a controller. The catheter includes one or more electrodes and also includes a transducer that can be controlled to emit a selective amount of energy. The catheter is configured such that at least a distal portion of the catheter is insertable into a biological lumen that is surrounded by tissue including nerves that are to be denervated using the transducer of the catheter. The excitation source is configured to selectively provide energy to the transducer of the catheter. The sensing subsystem is electrically coupled to at least one of the one or more sensing electrodes of the catheter. The controller is communicatively coupled to the excitation source, the sensing subsystem, and the memory. The controller is configured to cause the excitation source to energize the transducer of the catheter to emit a first amount of energy that is sufficient to heat the nerves in the tissue surrounding the biological lumen to an extent that a neural response is evoked without denervating the nerves. The controller is also configured to cause the sensing subsystem to sense, using at least one of the one or more electrodes of the catheter, the neural response that is evoked by energizing the transducer to emit the first amount of energy. Additionally, the controller is configured to determine one or more characteristics of the sensed neural response of the nerves within the tissue surrounding the biological lumen, the one or more characteristics indicative of one or more of a size, type, function or health of the nerves and/or indicative of proximity of the nerves relative to the at least one of the one or more electrodes of the catheter. The controller is also configured to select, based on the one or more characteristics of the sensed neural response, one or more denervation parameters to be used for energizing the transducer of the catheter to emit a second amount of energy that is sufficient to denervate at least some the nerves in the tissue surrounding the biological lumen. The controller is further configured to use the selected one or more denervation parameters to cause the excitation source to energize the transducer of the catheter to emit the second amount of energy that is sufficient to denervate at least some the nerves in the tissue surrounding the biological lumen.

This summary is not intended to be a complete description of the embodiments of the present technology. Other features and advantages of the embodiments of the present technology will appear from the following description in which the preferred embodiments have been set forth in detail, in conjunction with the accompanying drawings and claims.

The details of one or more examples of the invention are set forth in the accompanying drawings and the description below. Other features and advantages will be apparent from the description and drawings, and from the claims.

### BRIEF DESCRIPTION OF THE FIGURES

FIGS. 1A and 1B are high level flow diagrams used to summarize methods according to certain embodiments of the present technology.

FIG. 2 includes a table showing how nerve denervation and tissue necrosis are dependent on the duration that tissue is heated to at least 43 degrees Celsius.

FIG. 3A shows an example catheter with its two selectively deployable electrodes in their non-deployed positions.

FIG. 3B shows the catheter, which was introduced in FIG. 3A, with its two selectively deployable electrodes in their deployed positions.

FIG. 4 is a schematic diagram of an example system, according to an embodiment of the present technology, for interfacing with a patient's arterial nerves.

FIGS. 5A and 5B illustrate example cross-sections of a portion of the shaft of the catheter shown in FIGS. 3A and 3B.

FIG. 6 illustrates example details of a fluid supply subsystem introduced in FIG. 4.

FIGS. 7A and 7B illustrates, respectively, a longitudinal cross-sectional view and a radial cross-sectional view of an example transducer of the catheter shown in FIGS. 3A and 3B.

### DETAILED DESCRIPTION

In the following detailed description of example embodiments, reference is made to specific example embodiments by way of drawings and illustrations. These examples are described in sufficient detail to enable those skilled in the art to practice what is described, and serve to illustrate how elements of these examples may be applied to various purposes or embodiments. Other embodiments exist, and logical, mechanical, electrical, and other changes may be made. Features or limitations of various embodiments described herein, however important to the example embodiments in which they are incorporated, do not limit other embodiments, and any reference to the elements, operation, and application of the examples serve only to define these example embodiments. Features or elements shown in various examples described herein can be combined in ways other than shown in the examples, and any such combination is explicitly contemplated to be within the scope of the examples presented here. The following detailed description does not, therefore, limit the scope of what is claimed.

Regulating operation of the nervous system to characterize nerve signaling and modulate organ function includes in some examples introduction of a catheter (aka a probe) into the body to a specified anatomical location (e.g., within a biological lumen, such as a renal artery, but not limited thereto), and at least partially destroying or ablating nerves using the probe to destroy nerve tissue in the region near the probe. By reducing nerve function in the selected location, an abnormally functioning physiological process can often be regulated back into a normal range. It would also be possible to modulate nerve function to purposely cause an abnormally functioning that is beneficial to the patient.

Unfortunately, it is typically difficult to estimate the degree to which nerve activity should be or has been reduced, which makes it difficult to perform a denervation procedure where it is desired to ablate all nerves, or to ablate some, but not all, nerves to bring the nervous system response into a desired range without destroying the nervous system response entirely.

As noted above, when a denervation procedure is performed, preferred denervation parameters (which can also be referred to as ablation parameters) to use for performing the denervation procedure on a specific patient are typically unknown. Rather, default denervation parameters are likely to be used during a denervation procedure, which may lead to an inefficient and/or ineffective procedure.

A denervation procedure may be used, for example, to perform renal nerve ablation (aka renal denervation) to treat hypertension. Various studies have confirmed that renal nerve activity has been associated with hypertension, and that ablation of the renal nerves can improve renal function and reduce hypertension. In a typical procedure, a catheter is introduced into a hypertensive patient's arterial vascular system and advanced into the renal artery. Renal nerves located in the arterial wall and in regions adjacent to the artery are ablated by destructive means such as RF waves, microwave, cryotherapy, ultrasound, laser or chemical agents to limit the renal nerve activity, thereby reducing hypertension in the patient.

As noted above, in order to determine what parameters should be used to perform a denervation procedure prior to the denervation procedure being performed, or whether additional denervation needs to be performed following a procedure, a neural response may need to be evoked. It is also possible to evoke a neural response in the first place, for the purpose of determining whether or not a denervation procedure should be performed in the first place in general and/or at a specific location along a biological lumen. One way to evoke a neural response is by delivering electrical stimulation to nerves in tissue surrounding a biological lumen. Previously it has been proposed that neural response of nerves surrounding a biological lumen (e.g., the renal artery) can be assessed by delivering electrical stimulation to the nerves before and after an ablation process is performed, to evoke a neural response to thereby assess the efficacy of the ablation process, which in turn can be used to determine whether one or more further ablation processes need to be performed. More specifically, it has been proposed that a probe be inserted into an artery and used to emit an electrical stimulus into the arterial lumen wall, and that a resulting evoked neural response can be sensed using the probe and stored as a baseline measurement, since no destruction to the nerves has yet been applied. After recording of the baseline measurement, an ablation process is performed to attempt to destroy certain nerves, such as renal nerves that surround the renal artery. The ablation process can be performed, e.g., using radio frequency (RF), microwave, or ultrasound energy to heat the nerves sufficiently such that they are denervated or destroyed. After the ablation process ceases, further electrical stimulus is emitted and a post-ablation resulting evoked neural response is sensed using the probe. The pre-ablation and post-ablation evoked neural responses are then compared to one another to assess the extent of the nerve destruction, and to determine whether additional ablation is appropriate, or whether sufficient destruction has been performed. It is noted that the terms ablate and denervate are used interchangeably herein. Similarly, the terms ablation and denervation are used interchangeably herein.

If a first probe is used to electrically evoke and measure neural responses, and a second probe is used to perform the ablation process, then a clinician needs to swap the first and second probes in and out of the biological lumen one or more times, which can be time consuming. Alternatively if a same probe is used to electrically evoke and measure a neural response during a first period of time, to perform an ablation process during a second period of time (that follows the first period of time), and then to electrically evoke and measure a neural response during a third period of time (that follows the second period of time), then the same probe may include at least a first pair of electrodes for delivering the electrical stimulus to evoke the neural response, a second pair of electrodes for sensing the evoked neural response, as well as the transducer that produces the ablation energy, e.g., an RF transducer (which can be electrodes), a microwave transducer, or an ultrasound transducer. Additionally, such a probe may include an expandable balloon that receives a circulating cooling fluid that can protect an inner wall of the biological lumen (e.g., a renal artery) in which the probe is located by transferring heat away from the treatment area (e.g., to ensure that the temperature of the transducer and/or adjacent tissue does not exceed a particular threshold). Including all of the aforementioned components on the same probe can increase probe production complexity and costs.

In accordance with certain embodiments of the present technology, rather than using a pair of electrodes on a probe to electrically evoke a neural response to electrical stimulation that is sensed by a further pair of electrodes, the transducer that is used to produce the ablation energy (aka denervation energy) does double duty as the mechanism that evokes a neural response. More specifically, rather than the neural response being invoked in response to an electrical stimulus, the neural response is instead invoked in response to the transducer (e.g., the RF, microwave, or ultrasound transducer) being used to sufficiently heat the nerves in the tissue surrounding the biological lumen to an extent that a neural response is evoked without denervating the nerves. Such an embodiment can reduce the number of electrodes that are included on the probe, thereby reducing the probe production complexity and costs. More specifically, in accordance with certain embodiments of the present technology, the same transducer that is used for performing ablation (aka denervation) is also used to evoke a neural response prior to (and/or after) ablation is performed, by using the transducer to sufficiently heat the nerves in the tissue surrounding the biological lumen to an extent that a neural response is evoked without denervating the nerves. This could make a catheter less complex by eliminating the need for stimulation electrodes to evoke the neural response. Indeed it is possible that the catheter includes only a single sensing electrode that is used for sensing an evoked neural response together with another electrode that is not part of the catheter.

The evoked response, that results from using the transducer (e.g., the RF, microwave, or ultrasound transducer) to sufficiently heat the nerves in the tissue surrounding the biological lumen to an extent that a neural response is evoked without denervating the nerves, can be due to heat simply exciting the nerves, or can be due to the heat causing a heat pain response (i.e., a pain response to heat stimuli).

In accordance with certain embodiments of the present technology, instead of (or in addition to) performing stimulation using electrical energy to evoke a neural response, the neural response can be evoked by heating nerves (sufficient to evoke a neural response without denervating the nerves) to sense a baseline neural response (prior to ablation), and/or to sense a post-ablation neural response to determine whether denervation was successful or if additional denervation needs to be performed.

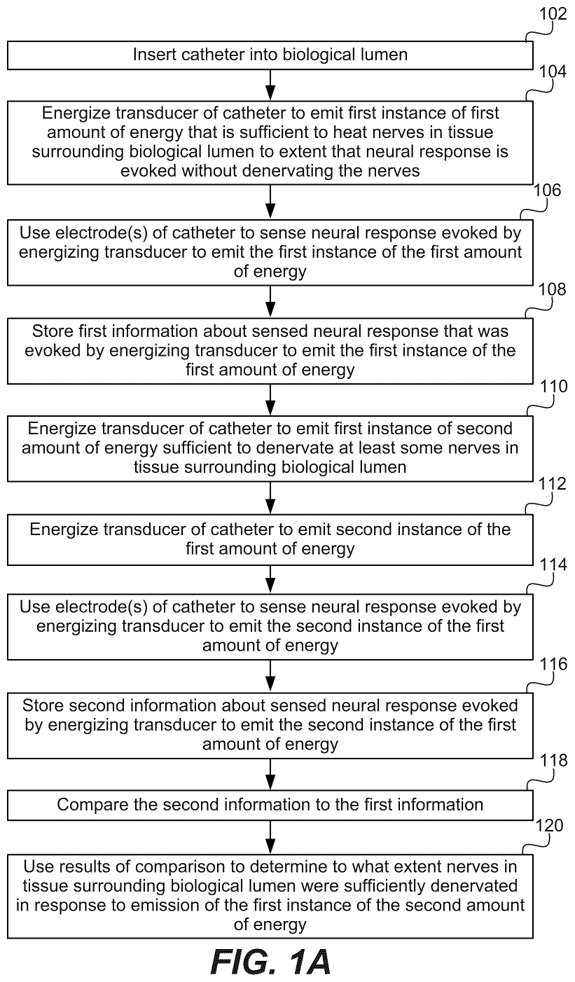

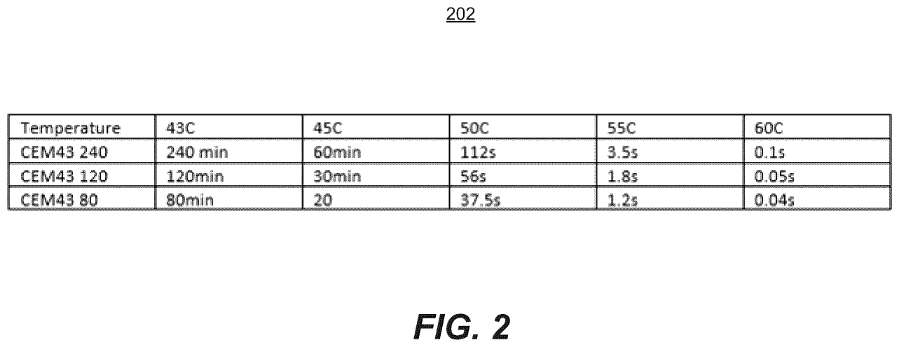

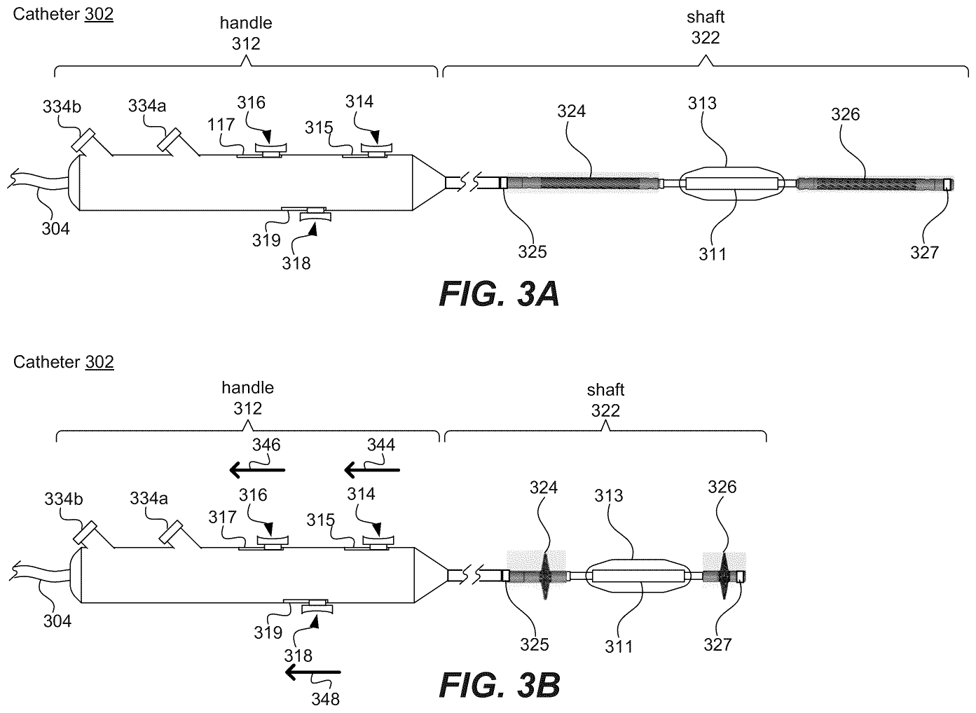

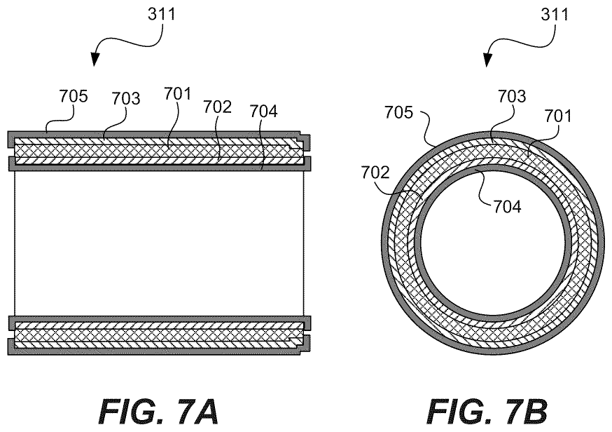

The high level flow diagram of FIG. 1A is now used to summarize methods according to certain embodiments of the present technology, wherein such methods are for use with a catheter including one or more electrodes that can be used to sense neural activity (e.g., an evoke neural response) and also including a transducer that can be controlled to emit a selective amount of energy. An example of such a catheter is described below with reference to FIGS. 3A and 3B. However, it is noted that the methods described with reference to FIG. 1A (and FIG. 1B) can alternatively be used with other catheters. The transducer that can be controlled to emit a selective amount of energy can be, e.g., an ultrasound transducer, a radio frequency (RF) transducer, or a microwave transducer, but is not limited thereto. More generally, the transducer that is used to perform a method described with reference to FIG. 1A (and FIG. 1B) should be capable of selectively heating tissue to at least 43 degrees Celsius, and preferably at least 50 degrees Celsius. Additionally, the duration and/or power level of energy emitted by the transducer that is used to perform a method described with reference to FIG. 1A (and FIG. 1B) should be capable of being controlled. Where the transducer is an ultrasound transducer, the transducer can be a piezoelectric transducer, but is not limited thereto. Examples of a piezoelectric ultrasound transducer are described below, e.g., with reference to FIGS. 3A, 3B and 7. Where the transducer is an RF transducer, the RF transducer can be implemented using one or more electrodes, but is not limited thereto.

Referring to FIG. 1A, step 102 involves inserting a catheter into a biological lumen (e.g., a renal artery), such that at least a distal portion of the catheter is inserted into the biological lumen, wherein the biological lumen (e.g., a renal artery) is surrounded by tissue including nerves (e.g., renal nerves) that are to be denervated using the transducer of the catheter. As noted above, an example of the catheter that can be inserted into a biological lumen at step 102, and used in other steps of FIG. 1A, is the catheter described below with reference to FIGS. 3A and 3B. But as also noted above, other types of catheters can alternatively be used, and embodiments of the present technology described herein are not limited to use with the example catheter described below with reference to FIGS. 3A and 3B. Indeed, as noted above, it is possible that the catheter includes only a single sensing electrode that is used for sensing an evoked neural response together with another electrode that is not part of the catheter.

Step 104 involves energizing the transducer of the catheter to emit a first instance of a first amount of energy that is sufficient to heat the nerves in the tissue surrounding the biological lumen to an extent that a neural response is evoked without denervating the nerves. In accordance with certain embodiments, the first amount of energy should be sufficient to heat tissue to about 43 degrees Celsius for a duration between about 0.5 seconds to about 2.0 seconds, in order to evoke a neural response without denervating the nerves. In accordance with other embodiments, the first amount of energy should be sufficient to heat tissue to about 50 degrees Celsius for a duration between about 0.5 seconds to about 2.0 seconds, in order to evoke a neural response without denervating the nerves. The term “about”, as used herein, means within plus and minus 10 percent of a specified value. More generally, in accordance with certain embodiments, the first amount of energy should be sufficient to heat tissue to a temperature within the range of 43 degrees Celsius to 53 degrees Celsius for a duration between about 0.5 seconds to about 2.0 seconds, in order to evoke a neural response without denervating the nerves.

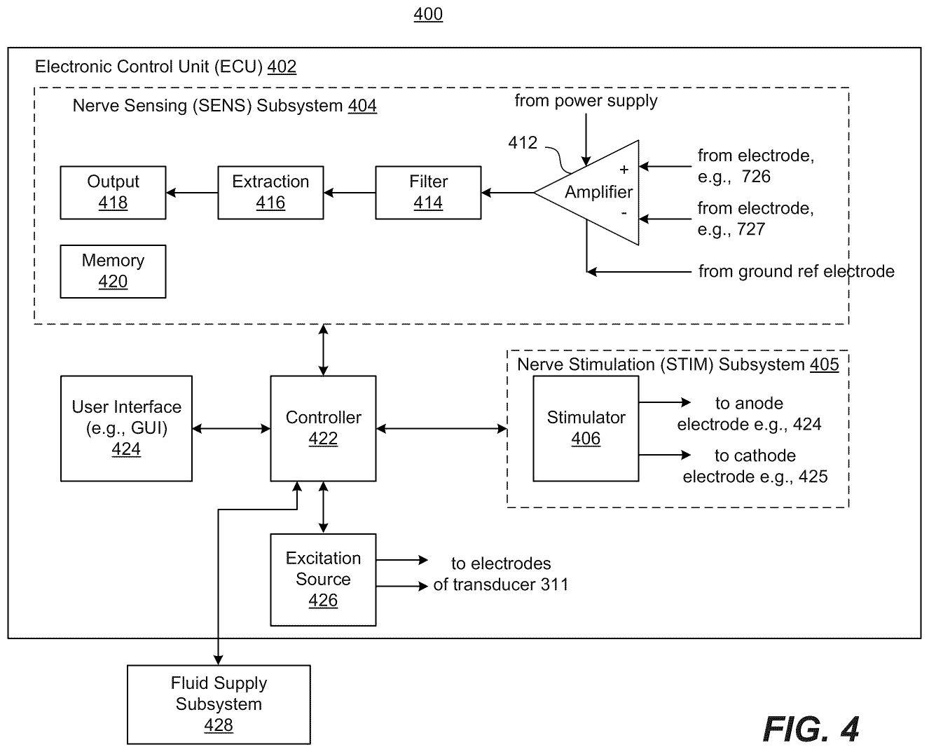

Step 106 involves using at least one of the one or more electrodes of the catheter to sense the neural response that is evoked by energizing the transducer to emit the first instance of the first amount of energy (at step 104), and step 108 involves storing first information about the sensed neural response (sensed at step 106) that was evoked by energizing the transducer to emit the first instance of the first amount of energy (at step 104). Step 106 is preferably performed while the transducer of the catheter remains at a same location within the biological lumen as when step 104 was performed. While steps 106 and 108 are shown as separate steps in FIG. 1A, they can be performed substantially contemporaneously and thus can be considered part of a same step. Additionally, step 104 can be performed substantially contemporaneously with steps 106 and 108. The information that is stored at step 108 can be stored, e.g., in memory of an Electronic Control Unit (ECU) of a tissue treatment system. Examples of such a system and components thereof are described below, e.g., with reference to FIG. 4.

Still referring to FIG. 1A, step 110 involves energizing the transducer of the catheter to emit a first instance of a second amount of energy that is sufficient to denervate at least some the nerves in the tissue surrounding the biological lumen. Step 110 is preferably performed while the transducer of the catheter remains at the same location within the biological lumen as when steps 104 and 106 were performed. Additional details of step 110, according to certain embodiments of the present technology, are described below.

Step 112 involves energizing the transducer of the catheter to emit a second instance of the first amount of energy. As noted above, the first amount of energy should be sufficient to heat the nerves in the tissue surrounding the biological lumen to an extent that a neural response is evoked without denervating the nerves. Step 112 is preferably performed while the transducer of the catheter remains at a same location within the biological lumen as when steps 104, 106, and 110 were performed. Step 114 involves using at least one of the one or more electrodes of the catheter to sense the neural response that is evoked by energizing the transducer to emit the second instance of the first amount of energy (at step 112). Step 114 is preferably performed while the transducer of the catheter remains at the same location within the biological lumen as when steps 104, 106, 110, and 112 were performed. Step 116 involves storing second information about the sensed neural response (sensed at step 114) that is evoked by energizing the transducer to emit the second instance of the first amount of energy (at step 112). While steps 114 and 116 are shown as separate steps in FIG. 1A, they can be performed substantially contemporaneously and thus can be considered part of a same step. Additionally, step 112 can be performed substantially contemporaneously with steps 114 and 116.

Step 118 involves comparing the second information (stored at step 116) to the first information (stored at step 108), and step 120 involves using results of the comparing (at step 118) to determine to what extent the nerves in the tissue surrounding the biological lumen were denervated in response to the emission of the first instance of the second amount of energy. The result of step 120 could be that sufficient denervation has been achieved, and thus, the denervation procedure (at least at the present position of the catheter within the biological lumen) is complete. Alternatively, the results of step 120 could be that sufficient denervation has not yet been achieved, and thus, that additional denervation should be performed. When additional denervation should be performed, such additional denervation is preferably performed while the transducer of the catheter remains at the same location within the biological lumen as when steps 104, 106, 110, 112, and 114 were performed.

In accordance with certain embodiments, the first information about the sensed neural response (sensed at step 106, and stored at step 108), and the second information about the sensed neural response (sensed at step 114, and stored at step 116), can be compared to one another to determine an amount (e.g., percent, or magnitude, but not limited thereto) that the neural response was reduced. The amount by which the neural response was reduced can then be compared to a corresponding threshold for the purpose of determining whether sufficient denervation has been achieved, or whether additional denervation should be performed. For example, if the neural response was reduced by at least X percent (e.g., 50%, 60%, or 80%, but not limited thereto), then it can be determined that sufficient denervation has been achieved.

In accordance with certain embodiments, in response to determining that the nerves in the tissue surrounding the biological lumen were not sufficiently denervated in response to the emission of the first instance of the second amount of energy, the method further comprises energizing the transducer of the catheter to emit a second instance of the second amount of energy. In other words, additional denervation could be performed, e.g., by energizing the transducer of the catheter to emit a second instance of the second amount of energy that is sufficient to denervate at least some the nerves in the tissue surrounding the biological lumen. As noted above, when additional denervation is performed, such additional denervation is preferably performed while the transducer of the catheter remains at the same location within the biological lumen as when steps 104, 106, 110, 112, and 114 were performed.

In accordance with other embodiments, in response to determining that the nerves in the tissue surrounding the biological lumen were not sufficiently denervated in response to the emission of the first instance of the second amount of energy, the method includes energizing the transducer of the catheter to emit a third amount of energy that is greater than (or less than) the second amount of energy. In other words, additional denervation could be performed by energizing the transducer of the catheter to emit a first instance of the third amount of energy that is sufficient to denervate at least some the nerves in the tissue surrounding the biological lumen. The third amount of energy can be either less than or greater than the second amount of energy. The amount of energy delivered using the transducer can be a function of various factors, such as the level of the energy and/or the duration of the energy. It is also possible that the amount of energy delivered using the transducer can be a function of the frequency of the energy.

In accordance with certain embodiments, steps 104 and 106 are performed at the same time, i.e., contemporaneously. Assume, for example, that the transducer of the catheter is an ultrasound transducer. The frequency of the ultrasound energy emitted from such an ultrasound transducer would likely be in the high MHz range, while the evoked neural response would be much lower, e.g., in 0.1 to 2 kHz range. Thus, the emitted ultrasound energy signals and the sensed evoked response neural signals are in very distinctive frequency bands, which means neural sensing can be performed during ultrasound ablation. A lowpass filter or a bandpass filter with a cutoff at about 2-3 kHz up to about 2 MHz can be very effective in suppressing the potential ultrasound interference. RF frequencies and microwave frequencies are also much higher than the frequencies of evoked neural responses, and thus, are similarly readily distinguishable from the evoked neural response using a filter, thereby enabling steps 104 and 106 to be performed contemporaneously. For similar reasons to those just described above, steps 110 and 112 can also be performed at the same time, i.e., contemporaneously. As explained above, in certain embodiments the transducer of the catheter remains at the same location within the biological lumen when steps 104, 106, 110, 112, and 114 are performed

In certain embodiments, the neural response sensed at step 106 can be a pre-denervation baseline neural response and the first information stored at step 108 can be information about the pre-denervation baseline neural response. In certain such embodiments, the neural response sensed at step 114 can be a post-denervation neural response and the second information stored at step 116 can be information about the post-denervation neural response.

Still referring to FIG. 1A, at steps 104 and 112, the first amount of energy emitted from the transducer (that is sufficient to heat the nerves in the tissue surrounding the biological lumen to the extent that a neural response is evoked without denervating the nerves), is provided by causing the transducer to emit energy within a specified frequency range for a first duration and having a first power level that are collectively sufficient to heat the tissue surrounding the biological lumen to a temperature of at least 38 degrees Celsius, but not to exceed 52 degrees Celsius. In certain embodiments, at step 110, the second amount of energy emitted from the transducer (that is sufficient to denervate at least some the nerves in the tissue surrounding the biological), is provided by causing the transducer to emit energy within the same specified frequency range for a second duration and having a second power level that are collectively sufficient to heat the tissue surrounding the biological lumen to a temperature of at least 43 degrees Celsius, but not to exceed 100 degrees Celsius, wherein the second duration is greater than the first duration and/or the second power level is greater than the first power level. As will be described in further detail below, the specified frequency range that is used depends on the type of transducer.

In accordance with certain embodiments, the transducer of the catheter is an ultrasound transducer. In certain such embodiments, the specified frequency range of the energy emitted by the ultrasound transducer can be from 5 MHz to 20 MHz, and preferably is within the range of 8.5 MHz to 9.5 MHz. In certain such embodiments, the parameter(s) selected and used at steps 104, 110 and 112 (e.g., by the controller 422) to energize the ultrasound transducer can specify an amplitude, power, duration, frequency, and/or duty cycle of the ultrasound energy. In certain embodiments, the ultrasound transducer (e.g., 311) is located within a balloon (e.g., 313) that is at least partially filled with a cooling fluid (e.g., 613) that is circulated through the balloon in order to cool at least a portion of the tissue surrounding the biological lumen proximate the balloon, e.g., as described below with reference to FIGS. 3A through 6. In certain such embodiments, the denervation parameter(s) can specify a flow rate and/or a temperature associated with the cooling fluid.

In accordance with other embodiments, the transducer of the catheter is an RF transducer. Such an RF transducer can be implemented using one or more electrodes. In certain such embodiments, the specified frequency range of the energy emitted from the RF transducer is from 300 kHz to 600 kHz. In certain such embodiments, the parameter(s) selected and used at steps 104, 110 and 112 (e.g., by the controller 422) to energize the RF transducer can specify an amplitude, power, duration, frequency, and/or duty cycle of the RF energy.

In accordance with other embodiments, the transducer of the catheter is a microwave transducer. In certain such embodiments, the specified frequency range of the energy emitted from the microwave transducer is from 300 MHz to 30 GHz, and preferably is within the range of 900 MHz to 3 GHz. In certain such embodiments, the parameter(s) selected and used at steps 104, 110 and 112 (e.g., by the controller 422) to energize the microwave transducer can specify an amplitude, power, duration, frequency, and/or duty cycle of the microwave energy at step 110 and/or following step 120.

In accordance with certain embodiments of the present technology, characteristics of the evoked neural response sensed at step 106, and/or the evoked neural response sensed at step 114, can be quantified and used to tailor patient specific parameters of nerve destruction that is to be performed as part of a medical procedure. In other words, the evoked neural response, which is caused by energizing the transducer of the catheter to energy that is sufficient to heat nerves in tissue surrounding biological lumen to extent that neural response is evoked without denervating the nerves, can be used to determine (aka select) parameters of the second amount of energy (used at step 110) that is sufficient to denervate at least some nerves in tissue surrounding biological lumen. For example, between steps 106 and 110, one or more additional steps can be performed to determine parameters of the second amount of energy to be used at step 110. This can involve determining characteristics of the evoked neural response sensed at step 106, wherein such characteristics can include or be based on amplitudes of the multiple peaks and/or based on temporal spacings between the multiple peaks of the sensed signal indicative of the evoked neural activity. For example, an average amplitude of the multiple peaks can be determined and one or more denervation parameters can be selected (e.g., by the controller 422) based on the average amplitude of the multiple peaks. Where the average amplitude of the multiple peaks is relatively large, e.g., above a specified threshold (e.g., of 2.0 μV), that means there is relatively high neural activity that is sensed and that a relatively high amplitude and/or relatively long duration denervation therapy should be selected and used at step 110. Where the average amplitude of the multiple peaks is relatively small, e.g., below a specified threshold (e.g., of 2.0 μV), that means there is relatively low neural activity that is sensed and that a relatively low amplitude and/or relatively short duration denervation therapy should be selected (e.g., by the controller 422) and used at step 110. For a more specific example, once the average amplitude of the multiple peaks is below a specified threshold (e.g., of 2.0 μV), the amplitude and/or duration of the denervation therapy can be decreased by 50% compared to when the median amplitude of the multiple peaks is above the specified threshold.

Alternatively, or additionally, a median amplitude of the multiple peaks can be determined and denervation parameters can be selected based on the median amplitude of the multiple peaks. For example, a median amplitude of the multiple peaks can be determined and one or more denervation parameters can be selected (e.g., by the controller 422) based on the average amplitude of the multiple peaks. Where the median amplitude of the multiple peaks is relatively large, e.g., above a specified threshold (e.g., of 2.0 μV), that means there is relatively high neural activity that is sensed and that a relatively high amplitude and/or relatively long duration denervation therapy should be selected and used at step 110. Where the median amplitude of the multiple peaks is relatively small, e.g., below a specified threshold (e.g., of 2.0 μV), that means there is relatively low neural activity that is sensed and that a relatively low amplitude and/or relatively short duration denervation therapy should be selected (e.g., by the controller 422) and used at step 110. For a more specific example, once the median amplitude of the multiple peaks is below a specified threshold (e.g., of 2.0 μV), the amplitude and/or duration of the denervation therapy can be decreased by 50% compared to when the median amplitude of the multiple peaks is above the specified threshold.

Alternatively, or additionally, determining one or more characteristics of the evoked neural response sensed at step 106 (e.g., by the controller 422) can involve fitting a curve to a portion of the signal indicative of the neural activity, and determining an area under the curve, and step 108 can involve the selecting denervation parameters based on the area under the curve. Where the area under the curve is relatively large, e.g., above a specified threshold, that means there is relatively high neural activity that is sensed and that a relatively high amplitude and/or relatively long duration denervation therapy should be selected (e.g., by the controller 422) and used at step 110. Where the area under the curve is relatively small, e.g., below a specified threshold, that means there is relatively low neural activity that is sensed and that a relatively low amplitude and/or relatively short duration denervation therapy should be selected (e.g., by the controller 422) and used at step 110. For a more specific example, once the area under the curve is below a specified threshold, the amplitude and/or duration of the denervation therapy can be decreased by 50% compared to when the area under the curve is above the specified threshold.

Where additional denervation energy is to be delivered following step 120, similar techniques to those just described above can be used to determine denervation parameters that are to be used following step 120.

Additionally details of how denervation parameters can be selected are described in commonly assigned U.S. patent application Ser. No. 18/182,821, titled USING CHARACTERISTICS OF NATIVE OR EVOKED SENSED NEURAL ACTIVITY TO SELECT DENERVATION PARAMETERS, filed Mar. 13, 2023, which is incorporated herein by reference.

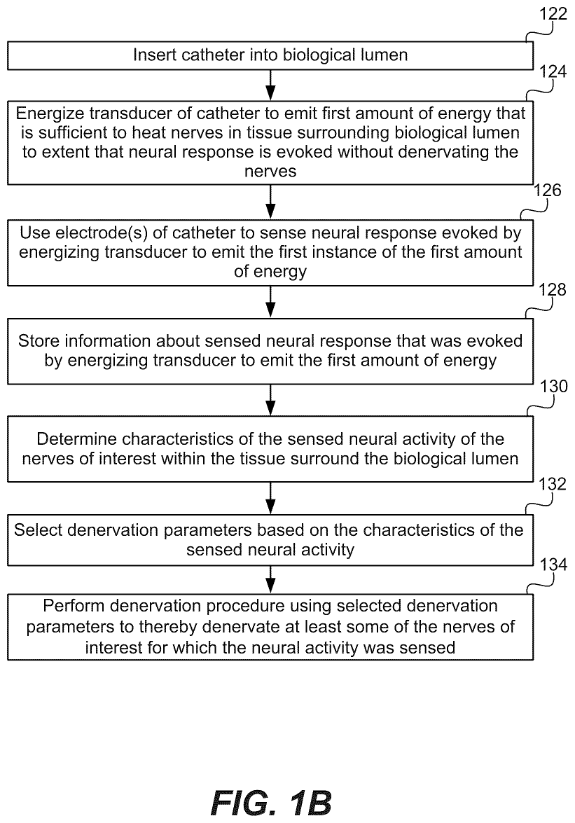

Regardless of what type of transducer is used, in certain embodiments a same frequency range is used at each of step 104, 110, and 112. A benefit of using the same frequency range to sufficiently heat the nerves in the tissue surrounding the biological lumen to the extent that a neural response is evoked without denervating the nerves (e.g., at step 104 and 112), as well as to sufficiently heat the nerves in the tissue surrounding the biological lumen to the extent that is sufficient to denervate at least some the nerves in the tissue surrounding the biological (e.g., at step 110), is that the same transducer can be used to achieve both affects. In such embodiments, it is the duration and/or power level of the emitted energy that can be adjusted to control whether or not denervation is achieved. For example, in certain embodiments, the same power level used at step 110 is also used at each of steps 104 and 112, so long as the duration of the energizing of the transducer at step 110 is greater than the duration of the energizing of the transducer as each of steps 104 and 112. This is because nerve denervation and tissue necrosis are dependent on the duration that tissue is heated to at least 43 degrees Celsius, as can be appreciated from the table 202 shown in FIG. 2. More generally, the amount of energy emitted by a transducer is dependent on both the power level and duration used to energize the transducer. In other embodiments, the power level used at step 110 is greater than is used at each of steps 104 and 112, in which case the duration of the energizing of the transducer at step 110 could be the same as the duration of the energizing of the transducer at each of steps 104 and 112. In further embodiments, the power level used at step 110 is greater than is used at each of steps 104 and 112, and the duration of the energizing of the transducer at step 110 is greater than the duration of the energizing of the transducer at each of steps 104 and 112.

FIG. 1B is a high level flow diagram used to summarize methods according to certain embodiments of the present technology. Referring to FIG. 1B, step 122, 124, 126 and 128 are respectively the same as, or very similar to, respectively, step 102, 104, 106 and 108 described above with reference to FIG. 1A. Accordingly, details of steps 122, 124, 126 and 128 need to be provided, since reference can be made back to the description of FIG. 1A. Still referring to FIG. 1B, step 130 involves determining one or more characteristics of the sensed neural activity of the nerves within the tissue surround the biological lumen, wherein the characteristic(s) is/are indicative of one or more of the size, type, function or health of the nerves for which neural activity is sensed and/or indicative of proximity of the nerves relative to the sensing electrode(s) of the catheter, but not limited thereto. In certain embodiments, step 130 can be performed by a controller 422 of the system 400 described below with reference to FIG. 4.

Step 132 involves selecting one or more denervation parameters based on the characteristic(s) of the sensed neural activity, wherein the denervation parameter(s) is/are for use in performing a denervation procedure intended to denervate at least some of the nerves for which the neural activity was sensed. In certain embodiments, step 132 is performed by the controller 422 of the system 400 described below with reference to FIG. 4. In accordance with certain embodiments, the selecting of denervation parameter(s) at step 132 is performed using one or more processors, e.g., of an electrical control unit (ECU), an example of which is described below with reference to FIG. 4. Such an ECU can include a controller (e.g., 422 in FIG. 4), wherein the controller includes one or more processors. In certain embodiments, one or more denervation parameters is/are selected using one or more tables stored in memory (e.g., 420 in FIG. 4) of an ECU (e.g., 402 in FIG. 4) and accessed by at least one processor (e.g., of a controller 422 in FIG. 4). In other embodiments, one or more denervation parameters is/are selected using a machine learning model implemented by at least one of the one or more processors, or more generally, using artificial intelligence.

Step 134 involves performing the denervation procedure using the selected denervation parameter(s) to thereby denervate at least some of the nerves for which the neural activity was sensed. This step 132 can involve a controller (e.g., 422) controlling a signal generator (e.g., 406) to generate signals for performing the denervation procedure using the selected one or more denervation parameters. In accordance with certain embodiments, step 134 is performed using the same transducer used at step 124, except at step 134 the power level and/or duration used to energize the transducer is greater at step 134 compared to at step 124. More specifically, step 134 involves energizing the transducer of the catheter to emit an amount of energy that is sufficient to denervate at least some the nerves in the tissue surrounding the biological lumen. In accordance with certain embodiments, following step 134, additional steps that are the same as, or similar to, steps 112 through 120 (described above with reference to FIG. 1A) can be performed.

Example details were described above of how characteristics of the evoked neural response sensed at step 126 can be quantified and used to tailor patient specific parameters of nerve destruction that is to be performed as part of a medical procedure. As noted above, such characteristics can include or be based on amplitudes of the multiple peaks and/or based on temporal spacings between multiple peaks of the sensed signal indicative of the evoked neural response. For example, an average amplitude of the multiple peaks can be determined and one or more denervation parameters can be selected (e.g., by the controller 422) based on the average amplitude of the multiple peaks. Alternatively, or additionally, a median amplitude of the multiple peaks can be determined and denervation parameters can be selected based on the median amplitude of the multiple peaks. Alternatively, or additionally, a curve can be fit to a portion of the signal indicative of the neural activity, denervation parameters can be selected based on the area under the curve. Additionally details of how denervation parameters can be selected are described in commonly assigned U.S. patent application Ser. No. 18/182,821, titled USING CHARACTERISTICS OF NATIVE OR EVOKED SENSED NEURAL ACTIVITY TO SELECT DENERVATION PARAMETERS, filed Mar. 13, 2023, which was incorporated herein by reference above, and some details of which are provided below.

In certain embodiments, characteristic(s) of the sensed neural activity is/are determined at step 130 (e.g., by the controller 422) based on amplitudes of the multiple peaks and/or based on temporal spacings between the multiple peaks of the sensed signal indicative of the neural activity. For example, an average amplitude of the multiple peaks can be determined and one or more denervation parameters can be selected (e.g., by the controller 422) based on the average amplitude of the multiple peaks. Where the average amplitude of the multiple peaks is relatively large, e.g., above a specified threshold (e.g., of 2.0 μV), that means there is relatively high neural activity that is sensed and that a relatively high amplitude and/or relatively long duration denervation therapy should be selected at step 132 and used at step 134. Where the average amplitude of the multiple peaks is relatively small, e.g., below a specified threshold (e.g., of 2.0 μV), that means there is relatively low neural activity that is sensed and that a relatively low amplitude and/or relatively short duration denervation therapy should be selected (e.g., by the controller 422) at step 132 and used at step 134. For a more specific example, once the average amplitude of the multiple peaks is below a specified threshold (e.g., of 2.0 μV), the amplitude and/or duration of the denervation therapy can be decreased by 50% compared to when the median amplitude of the multiple peaks is above the specified threshold.

Alternatively, or additionally, a median amplitude of the multiple peaks can be determined and denervation parameters can be selected based on the median amplitude of the multiple peaks. For example, a median amplitude of the multiple peaks can be determined and one or more denervation parameters can be selected (e.g., by the controller 422) based on the average amplitude of the multiple peaks. Where the median amplitude of the multiple peaks is relatively large, e.g., above a specified threshold (e.g., of 2.0 μV), that means there is relatively high neural activity that is sensed and that a relatively high amplitude and/or relatively long duration denervation therapy should be selected at step 132 and used at step 134. Where the median amplitude of the multiple peaks is relatively small, e.g., below a specified threshold (e.g., of 2.0 μV), that means there is relatively low neural activity that is sensed and that a relatively low amplitude and/or relatively short duration denervation therapy should be selected (e.g., by the controller 422) at step 132 and used at step 134. For a more specific example, once the median amplitude of the multiple peaks is below a specified threshold (e.g., of 2.0 μV), the amplitude and/or duration of the denervation therapy can be decreased by 50% compared to when the median amplitude of the multiple peaks is above the specified threshold.

Alternatively, or additionally, determining one or more characteristics of the sensed neural activity at step 130 (e.g., by the controller 422) can involve fitting a curve to a portion of the signal indicative of the neural activity, and determining an area under the curve, and step 132 can involve the selecting denervation parameters based on the area under the curve. Where the area under the curve is relatively large, e.g., above a specified threshold, that means there is relatively high neural activity that is sensed and that a relatively high amplitude and/or relatively long duration denervation therapy should be selected at step 132 (e.g., by the controller 422) and used at step 134. Where the area under the curve is relatively small, e.g., below a specified threshold, that means there is relatively low neural activity that is sensed and that a relatively low amplitude and/or relatively short duration denervation therapy should be selected at step 132 (e.g., by the controller 422) and used at step 134. For a more specific example, once the area under the curve is below a specified threshold, the amplitude and/or duration of the denervation therapy can be decreased by 50% compared to when the area under the curve is above the specified threshold.

In each of the above embodiments, the denervation procedure can be considered complete when the average, median, or area under the curve is below a corresponding specified threshold. For example, in certain embodiments, where the average or median amplitude of multiple peaks is below 0.5 μV, then it can be concluded the neural activity is sufficient low such that no (or no further) denervation therapy is needed.

Each nerve that surrounds a biological lumen (for which neural activity is being sensed) will typically fire only once per cardiac cycle. Accordingly, where the temporal spacings between sensed peaks is small relative to a cardiac cycle length, that can be interpreted as there being many nerves firing during the cardiac cycle. By contrast, where the temporal spacing between sensed peak is large relative to a cardiac cycle length, that can be interpreted as there being relatively few nerves firing during the cardiac cycle. Such observations can be used to determine whether a denervation procedure, or a further denervation procedure, should be performed, and/or to select one or more denervation parameters.

When a nerve fiber is being ablated during a denervation procedure, such that the health of the nerve fibers has been diminished or the nerve has been destroyed, the amplitude and shape of a sensed signal indicative of the neural activity, its latency, and its synchrony will change, compared to such characteristics prior to the denervation procedure. In certain embodiments, one or more of these various characteristics, such as amplitude, shape, latency, and synchrony, are used to quantify a neural fiber's health.

In accordance with certain embodiments, convolution is used to determine how far nerve fibers are from a sensing electrode (e.g., 326, 327), or more generally from a sensing site (which can also be referred to as a recording site). Typically, the larger the amplitude of the sensed neural activity the more likely nerve fibers are close to the sensing site, and the smaller the amplitude of the sensed neural activity the more likely the nerve fibers are far from the sensing site. Assume for example the neural activity of two different nerve fibers are sensed using a sensing electrode on a catheter, and that one of the nerve fibers is relatively far away from the sensing site, while another one of the nerve fibers is relatively close to the sensing site. Also assume that both the relatively far and the relatively close nerve fibers fire at the same time in response to stimulation energy that is intended to evoke a neural response. When a catheter is used to sense the above described neural activity, characteristics of both the relatively far and the relatively near neural fibers can be distinguished from one another in a sensed signal, which can enable a system and/or physician to approximate how far the different neural fibers are from the sensing site, as well as the size of such fibers, which are examples of characteristics of sensed neural activity that can be determined (e.g., by the controller 422) at step 130 in FIG. 1B.

In certain embodiments, sensed neural activity following a denervation procedure is compared to baseline neural activity sensed prior to the denervation procedure to determine the efficacy of the denervation procedure. Based on such a comparison, there can be a determination of whether the denervation procedure was sufficiently successful such that it could be terminated, or whether further ablation energy should be applied because sufficient nerve destruction has not yet been achieved. After enough data has been collected from a patient population, it may also be possible to analyze neural activity from a patient without requiring any baseline measurements. However, until such sufficient patient population data is collected, determining baseline measurements prior to a procedure, and comparing those to post-procedure measurements, is likely a good way to quantify the efficacy of the denervation procedure and to determine whether additional denervation treatment is needed.

In certain embodiments, neural activity can be sensed (e.g., by the sensing circuit 404) and used (e.g., by the controller 422) to diagnose a disease state of a patient, such as to diagnose a patient as having hypertension, since a patient with hypertension will have a neural activity signature indicative of hypertension that is distinguishable from the neural activity signature of a healthy patient that is not experiencing hypertension.

In FIG. 2 the term CEM43 refers to the cumulative equivalent minutes at 43 degrees Celsius model. As can be appreciated from FIG. 2, heating tissue to 43 degrees Celsius for 240 minutes, is equivalent to heating tissue to: 45 degrees Celsius for 60 minutes, 50 degrees Celsius for 112 seconds, 55 degrees Celsius for 2.5 seconds, and degrees Celsius for 0.1 seconds. FIG. 2 also shows that heating tissue to 43 degrees Celsius for 120 minutes, is equivalent to heating tissue to: 45 degrees Celsius for 30 minutes, 50 degrees Celsius for 56 seconds, 55 degrees Celsius for 1.8 seconds, and 60 degrees Celsius for 0.05 seconds. Additionally, FIG. 2 shows that heating tissue to 43 degrees Celsius for 80 minutes, is equivalent to heating tissue to: 45 degrees Celsius for minutes, 50 degrees Celsius for 37.5 seconds, 55 degrees Celsius for 1.2 seconds, and 60 degrees Celsius for 0.04 seconds.

At each of steps 106 and 112, the electrode(s) of the catheter that is/are used to sense the neural response that is evoked by energizing the transducer to emit the first amount of energy, can be a pair of electrodes of the catheter itself, an electrode of the catheter and an electrode of a guidewire, an electrode of the catheter and an electrode of an introducer sheath, or an electrode of the catheter and an external skin electrode. Other variations are also possible and within the scope of the embodiments described herein.

In the embodiments summarized above with reference to FIG. 1A, the same transducer used at step 104 (to emit a first instance of a first amount of energy that is sufficient to heat the nerves in the tissue surrounding the biological lumen to an extent that a neural response is evoked without denervating the nerves), is thereafter used at step 110 (to emit a first instance of a second amount of energy that is sufficient to denervate at least some the nerves in the tissue surrounding the biological lumen), as well as at step 112 (to emit a second instance of the first amount of energy that is sufficient to heat the nerves in the tissue surrounding the biological lumen to an extent that a neural response is evoked without denervating the nerves). Accordingly, in such embodiments a same catheter can be used to perform all of steps 104, 110 and 112, eliminating the need for different catheters to be swapped in and out of the biological lumen. In alternative embodiments, the transducer used at step 110 is different than the transducer used at steps 104 and 112. The different transducer used at step 110 can be part of the same catheter that includes the transducer used at steps 104 and 112. Alternatively, the different transducer used at step 110 can be part of a different catheter than the catheter that includes that transducer used at steps 104 and 112, in which case at least two different catheters would need to be swapped in and out of the biological lumen to perform such an alternative method. The different transducers can be the same type of transducer, or can be different types of transducers, e.g., a first transducer can be an ultrasound transducer, and a second transducer can be an RF transducer, or vice versa, but not limited thereto. Such alternative methods are also within the scope of the embodiments described herein.

In the embodiments summarized with reference to FIG. 1B, the same transducer used at step 124 (to emit a first instance of a first amount of energy that is sufficient to heat the nerves in the tissue surrounding the biological lumen to an extent that a neural response is evoked without denervating the nerves), can thereafter be used at step 134 (to perform the denervation procedure using the selected denervation parameter(s) to thereby denervate at least some of the nerves for which the neural activity was sensed). Accordingly, in such embodiments a same catheter can be used to perform both of steps 124 and 134, eliminating the need for different catheters to be swapped in and out of the biological lumen. In alternative embodiments, the transducer used at step 134 is different than the transducer used at step 124. The different transducer used at step 124 can be part of the same catheter that includes that transducer used at steps 124. Alternatively, the different transducer used at step 134 can be part of a different catheter than the catheter that includes that transducer used at step 124, in which case, at least two different catheters would need to be swapped in and out of the biological lumen to perform such an alternative method. The different transducers can be the same type of transducer, or can be different types of transducers, e.g., a first transducer can be an ultrasound transducer, and a second transducer can be an RF transducer, or vice versa, but not limited thereto. Such alternative methods are also within the scope of the embodiments described herein.

In accordance with certain embodiments, the biological lumen referred to in the flow diagrams of FIGS. 1A and 1B is a renal artery and the nerves comprise renal nerves innervating a kidney. The method can also include removing any catheter (e.g., 302) that had been inserted into a biological lumen, such as a renal artery. Where a denervation procedure described herein is being performed using a catheter (e.g., 302) inserted into a renal artery type of biological lumen, the disease being treated using the denervation procedure can be hypertension or some other disorder associated with elevated sympathetic nerve activity, as can be appreciated from the above discussion. However, it is noted that embodiments of the present technology described herein can alternatively be used in the performance of denervation procedures using catheters that are inserted into other types of biological lumens, besides a renal artery, to treat other types of diseases besides hypertension. For example, such other types of biological lumens include a vein, a pulmonary artery, a vascular lumen, a celiac artery, a common hepatic artery, a proper hepatic artery, a gastroduodenal artery, a hepatic artery, a splenic artery, a gastric artery, a blood vessel, a nonvascular lumen, an airway, a sinus, an esophagus, a respiratory lumen, a digestive lumen, a stomach, a duodenum, a jejunum, a cancer tissue, a tumor, an intestine, and a urological lumen, but are not limited thereto. Examples of other types of diseases that can be treated using an embodiment of the present technology include pulmonary hypertension, diabetes, obesity, nonalcoholic fatty liver disease, heart failure, end-stage renal disease, digestive disease, cancers, tumors, pain, asthma or chronic obstructive pulmonary disease (COPD), but are not limited thereto.

Embodiments of the present technology can be implemented using various different catheter implementations, and various implementations of an electronic control unit (ECU), and thus, are not limited to use with any specific catheter, ECU, and/or system of which a catheter and/or ECU is a part. Nevertheless, for completeness, an example catheter, ECU and system that can be used to implement embodiments of the present technology are described below. More specifically, FIGS. 3A through 7B are used to describe an example catheter, ECU and system that can be used to implement embodiments of the present technology that were described above. Such a system can also be referred to as an apparatus herein.

### Example Catheter

FIG. 3A shows a catheter 302 with its selectively deployable electrodes 324 and 326 in their non-deployed positions. The catheter 302 includes a catheter handle 312 and a catheter shaft 322. In addition to including the selectively deployable electrodes 324 and 326, the catheter shaft 322 is also shown as including a non-deployable electrode 325 that is proximal to the selectively deployable electrode 324, and a non-deployable electrode 327 that is distal the selectively deployable electrode 326. The selectively deployable electrode 324 can also be referred to as the proximal selectively deployable electrode 324, or more succinctly as the proximal electrode 324, or even more succinctly as the electrode 324. The selectively deployable electrode 326 can also be referred to as the distal selectively deployable electrode 326, or more succinctly as the distal electrode 326, or even more succinctly as the electrode 326. The catheter shaft 322 can also be referred to more succinctly herein as the shaft 322. The catheter 302 can be a specific implementation of the catheter 302 shown in and discussed above with reference to FIG. 3.

The catheter 322 is also shown as including a balloon 313 positioned longitudinally between the electrodes 324 and 326, wherein the balloon 313 is selectively inflatable and deflatable. The balloon 313 can also be referred as a selectively inflatable balloon 313, a selectively deployable balloon 313, or more succinctly as a balloon 313. When the balloon 313 is deflated, it can also be referred to as being non-inflated or in its non-deployed position. When the balloon 313 is inflated, it can also be referred to as being in its deployed position. As will be described in additional detail below, the balloon 313 can be selectively inflated by injecting a fluid into the balloon 313, and the balloon 313 can be selectively deflated by removing the fluid from the balloon 313. The balloon 313 can be made of an electrically insulating material such as polyamide, polyethylene terephthalate, or thermoplastic elastomer. In specific embodiments the balloon 313 is made from nylon, a polyimide film, a thermoplastic elastomer (such as those marked under the trademark PEBAX™), a medical-grade thermoplastic polyurethane elastomers (such as those marketed under the trademark PELLETHANE™), pellethane, isothane, or other suitable polymers or any combination thereof, but is not limited thereto.

The catheter handle 312, which can also be referred to more succinctly as the handle 312, includes actuators 314, 316, and 318, which can be used to selectively deploy the electrodes 324, 326, as well as to adjust a longitudinal distance between the electrodes 324, 326, as will be described in additional detail below. The actuators 314, 316, and 318 are respectively slidable within slots 315, 317, and 319 in the handle 312, and thus, the actuators 314, 316, and 318 can also be referred to as sliders. The catheter handle 312 is also shown as including a fluidic inlet port 334a and a fluidic outlet port 334b.

A fluid (e.g., expelled from a pressure syringe) can enter a fluid lumen (in the catheter shaft 322), via the fluidic inlet port 334a of the catheter 302, and then enter and at least partially fill the balloon 313. Fluid can be drawn from the balloon 313 (e.g., using a vacuum syringe) through another fluid lumen (in the catheter shaft 322) and out the fluidic outlet port 334b of the catheter 302. In this manner, the fluid can be used to selectively inflate and selectively deflate the balloon 313. In certain embodiments, fluid can be simultaneously injected into and removed from the balloon 313 to thereby circulate the fluid through the balloon 313.

The catheter 302 can also be referred to as an intraluminal microneurography probe 302, or more succinctly, as a probe 302. A cable 304, which extends from a proximal portion of the handle 312, provides for electrical connections between the catheter 302 (and more specifically, the electrodes thereof) and an electrical control unit (ECU), an example of which is described below with reference to FIG. 4.

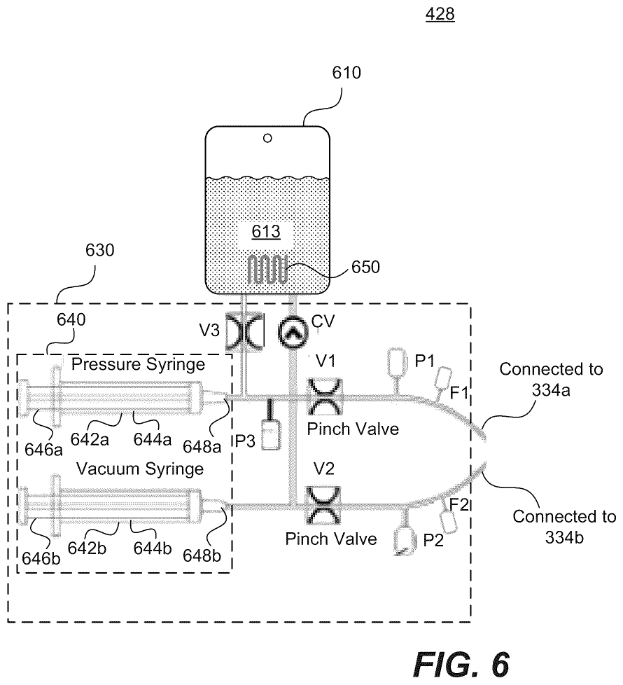

Still referring to FIG. 3A, a transducer 311 is shown as being within a balloon 313. The transducer 311 is an example of an ablation element that is included on the shaft 322 and configured to ablate nerve tissue using ultrasound energy. In other embodiments, the transducer and balloon may be replaced by a helical structure carrying a plurality of electrodes configured to deliver RF and/or pulsed electric field RF energy. In other embodiments, the transducer and balloon may be replaced by a microwave transmitting element within an expandable centering element. In other embodiments, the transducer may be replaced by a cryotherapeutic applicator. In other embodiments, the transducer and balloon may be replaced by an infusion needle configured to deliver an ablative chemical to the renal nerves.

Where a transducer 311 is within the balloon 313, the fluid that is circulated through the balloon 313 can be referred to as a cooling fluid that is used to cool the transducer 311 and/or to cool a portion of a biological lumen that the balloon 313 is within, and/or to cool the biological tissue surrounding the lumen. It is also possible that the catheter 302 is devoid of the transducer 311 or other ablative means, and that a separate catheter that includes a transducer or other ablative means is used to deliver ablation energy (e.g., at step 602 and 604 in FIG. 6). Where the catheter 302 is devoid of the transducer 311 or other ablative means, one or more electrodes of the catheter 302 can be used for sensing native neural activity. One or more electrodes of the catheter 302 can be used for delivering stimulation energy and one or more further electrodes of the catheter 302 can be used for sensing an evoked neural response to the stimulation energy.

When the catheter 302 is inserted into a biological lumen, such as an artery, vein or other vasculature, it is the distal portion of the catheter 302 (and more specifically the shaft 322) that is inserted into the biological lumen, and the proximal end of the catheter 302 (and more specifically the handle 312) that is used to maneuver the catheter 302. In the embodiment shown in FIGS. 3A and 3B, the electrode 326 can also be referred to as a distal selectively deployable electrode 326 as noted above, since it located closer to the distal end of the catheter 302 than to the proximal end of the catheter 302; and the electrode 324 can also be referred to as a proximal selectively deployable electrode 324 as noted above, since it is located closer to the proximal end of the catheter 302 than to the distal end of the catheter 302. For similar reasons, the electrode 325 can be referred to as the proximal non-deployable electrode 325, and the electrode 327 can be referred to as the distal non-deployable electrode 327.

FIG. 3B shows the catheter 302 with the electrodes 324 and 326 in their deployed (aka expanded) positions. In certain embodiments, the proximal selectively deployable electrode 324 is configured to be deployed (aka expanded) in response to the actuator 314 being slid in the proximal direction indicated by the arrow 344 in FIG. 3B. In such an embodiment, the proximal electrode 324 can be returned to its non-deployed (aka non-expanded or retracted) position in response to the actuator 314 being slid in the distal direction opposite the arrow 344 in FIG. 3B. More generally, the actuator 314 is used to selectively expand and retract the electrode 324.

In accordance with certain embodiments, the longitudinal distance between the distal electrode 326 and the proximal electrode 324 can be reduced by sliding the actuator 318 in the proximal direction indicated by the arrow 348 in FIG. 3B. Thereafter, the longitudinal distance between the distal electrode 326 and the proximal electrode 324 can be increased, if desired, by sliding the actuator 318 in the distal direction opposite the arrow 348 in FIG. 3B. More generally, the actuator 318 is used to adjust the longitudinal distance between the electrodes 324 and 326. The longitudinal distance between the proximal and distal electrodes 324, 326 can be any distance between the maximum and minimum longitudinal distance as controlled by a user using the actuator 318. In accordance with certain embodiments, the electrode 324 is configured to be deployed in response to the actuator 314 being slid in the proximal direction indicated by the arrow 344 in FIG. 3B. In accordance with certain embodiments, the distal electrode 326 is configured to be deployed in response to the actuator 316 being slid in the proximal direction indicated by the arrow 346 in FIG. 3B. In such an embodiment, the distal electrode 326 can be returned to its non-deployed position in response to the actuator 316 being slid in the distal direction opposite the arrow 346 in FIG. 3B. More generally, the actuator 316 is used to selectively expand and retract the electrode 326. Other variations are also possible and within the scope of the embodiment described herein.

Each of the selectively deployable electrodes 324, 326 can be made, for example, of a unitary nitinol tube that is laser cut to include apertures or openings having a predetermine pattern. In FIGS. 3A and 3B, each of the electrodes 324, 326 has laser cut spiral apertures that extend between proximal and distal portions of each of the electrodes 324, 326. The spiral apertures in each of the electrodes 324, 326 enable each of the electrodes to be selectively transitioned between their non-deployed and deployed positions. The apertures that are cut into the electrodes 324, 326 can have other shapes besides being spiral, so long as the apertures enable the electrodes to be transitioned between non-deployed and deployed positions. The selectively deployable electrodes 324, 326 can alternatively be mesh electrodes or spiral electrodes made of one or more electrically conductive wires, optionally with portions thereof being insulated. Other variations are also possible and within the scope of the embodiments described herein.

The catheter 302 can be configured to be introduced into a biological lumen, such as an artery, in a location near a body organ, such as a kidney. The catheter 302 can be introduced via an introducer sheath that is advanced to the intended catheter location in the biological lumen, and then withdrawn sufficiently to expose the shaft 322 to the biological lumen (e.g., renal artery). Once the shaft 322 is within the biological lumen, one of the electrodes 324, 326 can be deployed (aka expanded) using one of the actuators 314, 316 such that it is in contact with a portion of a circumferential interior wall of the biological lumen. The longitudinal distance between the electrodes 324 and 326 can then be adjusted, if desired, using the actuator 318. The other one of the electrodes 324, 326 can then be deployed (aka expanded) such that it is in contact with another portion of the circumferential interior wall of the biological lumen.

For example, where the catheter is inserted into a renal artery close to a kidney, the electrodes can be positioned near a nerve bundle that connects the kidney to the central nervous system, as the nerve bundle tends to approximately follow the artery leading to most body organs. The nerve bundle tends to follow the artery more closely at the end of the artery closer to the kidney, while spreading somewhat as the artery expands away from the kidney. As a result, it is desired in some examples that the catheter shaft 322 is small enough to introduce relatively near the kidney or other organ, as nerve proximity to the artery is likely to be higher nearer the organ.

Once the catheter 302 is in place, a practitioner can use instrumentation (e.g., the ECU 402) coupled to the electrodes to stimulate one or more nerves, and monitor for evoked nerve response signals used to characterize the nervous system response to certain stimulus. The transducer 311 and/or other ablative means are configured to ablate nerve tissue, such as by using ultrasound, RF, pulsed electric field RF, microwave, cryotherapy, or other energy or chemical means. Additionally, the catheter 302 can actively stimulate one or more nerves and sense resulting neural signals in between applications of ablation energy via the transducer 311, enabling more accurate control of the degree and effects of nerve ablation. In other examples, a catheter 302 lacking a transducer or other ablative means can be removed via a sheath, and an ablation probe (aka catheter) inserted, with the ablation probe removed and the catheter 302 reinserted to verify and characterize the effects of the ablation probe.

Any one or more electrodes of the catheter 302 can be selectively used to deliver stimulation energy to nerves surrounding a biological lumen. Similarly, any one or more electrodes of the catheter can be selectively used to sense neural activity of nerves surrounding a biological lumen, which can be spontaneous neural activity, or evoked neural activity.

For much of the below discussion, it is assumed that the transducer 311 is an ultrasound transducer that can be activated to deliver unfocused ultrasonic energy radially outwardly so as to suitably heat, and thus treat, tissue within the target anatomical region. The transducer 311 can be activated at a frequency, duration, and energy level suitable for treating the targeted tissue. In one nonlimiting example, the unfocused ultrasonic energy generated by the transducer 311 may target select nerve tissue of the subject, and may heat such tissue in such a manner as to neuromodulate (e.g., fully or partially ablate, necrose, or stimulate) the nerve tissue.

In accordance with certain embodiments, the transducer 311 includes a piezoelectric transducer body that comprises a hollow tube of piezoelectric material having an inner surface and an outer surface, with an inner electrode disposed on the inner surface of the hollow tube of piezoelectric material, and an outer electrode disposed on the outer surface of the hollow tube of piezoelectric material. In such embodiments, the hollow tube of piezoelectric material is an example of the piezoelectric transducer body. The hollow tube of piezoelectric material, or more generally the piezoelectric transducer body, can be cylindrically shaped and have a circular radial cross-section. However, in alternative embodiments the hollow tube of piezoelectric material can have other shapes besides being cylindrical with a circular radial cross-section. Other cross-sectional shapes for the hollow tube of piezoelectric material, and more generally the piezoelectric transducer body, include, but are not limited to, an oval or elliptical cross-section, a square or rectangular cross-section, pentagonal cross-section, a hexagonal cross-section, a heptagonal cross-section, an octagonal cross-section, and/or the like. The hollow tube of piezoelectric material, and more generally the piezoelectric transducer body, can be made from various different types of piezoelectric material, such as, but not limited to, lead zirconate titanate (PZT), polyvinylidene fluoride (PVDF), or other presently available or future developed piezoelectric ceramic materials. In other embodiments, the transducer 311 can be made of other materials and/or can have other shapes.

In certain embodiments, the transducer 311 is an ultrasound transducer configured to deliver acoustic energy in the frequency range of 8.5 to 9.5 MHz. In certain embodiments, the transducer is configured to deliver acoustic energy in the frequency range of 8.7-9.3 MHz or 8.695-9.304 MHz. Transducers delivering acoustic energy in the frequency range of 8.7-9.3 MHz have been shown to produce ablation up to mean depths of 6 mm. The piezoelectric transducer body is configured to generate ultrasonic waves in response to a voltage being applied between the inner and outer electrodes. One or both of the inner and outer electrodes can be covered by an electrical insulator to inhibit (and preferably prevent) a short circuit from occurring between the inner and outer electrodes when the ultrasound transducer is placed within an electrically conductive fluid and a voltage is applied between the inner and outer electrodes. Such an electrical insulator can be parylene, and more specifically, a parylene conformal coating, but is not limited thereto. An excitation source (e.g., 426 in FIG. 4) may be electrically coupled to inner and outer electrodes of the transducer 311, and may actuate the transducer 311 by applying a voltage between the inner and outer electrodes (or any other pair of electrodes), so as to cause the piezoelectric material of the piezoelectric transducer body to generate an unfocused ultrasonic wave that radiates radially outwardly.

### Example Electrical Control Unit (ECU)

FIG. 4 is a high level block diagram of an electrical control unit (ECU) 402 that is configured to be in electrical communication with a catheter, such as the catheter 302 described above. The ECU 402, and the catheter (e.g., 302) to which the ECU 402 is electrically coupled via a cable (e.g., 304), can be referred to more generally as a system 400. The ECU 402 can process a received signal to produce an output signal, and present information including information about the output signal, the received signal, or processing information. Such a system can be used, for example, in diagnostic procedures for assessing the status of a patient's nervous activity proximate a biological lumen, such as a vein or an artery, e.g., a renal artery, or another type of blood vessel. Such a system can be additionally, or alternatively, be used to select preferred denervation parameters for use in a denervation procedure. A same catheter that is used to assess the status of a patient's nervous activity proximate and/or select preferred denervation parameters can also be used to perform a denervation procedure.

Alternatively, it is possible that the catheter that is used to perform a denervation procedure differs from the catheter that is used to assess that status of a patient's nervous activity proximate a biological lumen, in which case different catheters can be swapped in and out of a biological lumen during a procedure.

Still referring to FIG. 4, the ECU 402 includes a stimulator 406 electrically coupled to a selected pair of electrodes (e.g., 324 and 325) of the catheter 302. The stimulator 406 which is part of a STIM circuit or subsystem 404, can selectively emit electrical signals (including stimulation pulses) having a specific voltage, amperage, duration, duty cycle and/or frequency of application that will cause nerve cell activation. For an example, the electrode 327 can be connected as the stimulation anode and the electrode 326 can be connected as the stimulation cathode, or vice versa. For another example, the electrode 324 can be connected as the stimulation anode and the electrode 325 can be connected as the stimulation cathode, or vice versa. Switches, which are not specifically shown, can be used to selectively control how various electrodes (e.g., 324, 325, 326 and 326) are coupled to various nodes of the ECU 402, such as to the input terminals of the amplifier 412, or to the output terminals of the stimulator 406. In this manner, the switches can be used to control which electrodes are configured as stimulation electrodes and which electrodes are configured as sensing electrodes.

Upon receiving the stimulation signal produced by the stimulator 406, the electrodes of the catheter 302 that are connected as stimulation electrodes (e.g., 324 and 325) can apply electrical energy to a patient's nerves through the biological lumen wall based on the received signal. Such stimulus can have any of a variety of known waveforms, such as a sinusoid, a square wave form or a triangular wave form, but is not limited thereto. In various examples, the stimulation can be applied for durations between approximately 0.05 milliseconds (msec) and approximately 8 msec.

The stimulation of nerves can be performed to evoke an elicited potential, which can cause such a potential to propagate in every direction along the nerve fibers. More generally, the STIM subsystem 405 can be used to deliver electrical stimulation via a selected pair of electrodes in order to evoke a neural response, and the SENS subsystem 404 can be used to sense the evoked neural response.

In some embodiments, the ECU 402 can digitally sample the signal sensed using a pair of electrodes to receive the electrical signal from the catheter 302. In alternate embodiments, the signal can be recorded as an analog signal. When receiving an electrical signal from the electrodes on the catheter 302, the ECU 402 can perform filtering and/or other processing steps on the signal. Generally, such steps can be performed to discriminate the signal sensed by the catheter from any background noise within the patient's vasculature such that the resulting output is predominantly the signal from nerve cell activation. In some instances, the ECU 402 can modulate the electrical impedance of the signal receiving portion in order to accommodate the electrical properties and spatial separation of the electrodes mounted on the catheter in a manner to achieve the highest fidelity, selectively and resolution for the signal received. For example, electrode size, separation, and conductivity properties can impact the field strength at the electrode/tissue interface.

Additionally or alternatively, the ECU 402 can comprise a headstage and/or an amplifier to perform any of offsetting, filtering, and/or amplifying the signal received from the catheter. In some examples, a headstage applies a DC offset to the signal and performs a filtering step. In some such systems, the filtering can comprise applying notch and/or band-pass filters to suppress particular undesired signals having a particular frequency content or to let pass desired signals having a particular frequency content. An amplifier can be used to amplify the entire signal uniformly or can be used to amplify certain portions of the signal more than others. For example, in some configurations, the amplifier can be configured to provide an adjustable capacitance of the recording electrode, changing the frequency dependence of signal pick-up and amplification. In some embodiments, properties of the amplifier, such as capacitance, can be adjusted to change amplification properties, such as the resonant frequency, of the amplifier.

In the illustrated embodiment of FIG. 4, the ECU 402 includes an amplifier 412 including a non-inverting (+) input terminal, an inverting (−) input terminal, a power supply input terminal, and a ground or reference terminal. As can be appreciated from FIG. 4, the non-inverting (+) input terminal can be coupled to the electrode 326, the inverting (−) input terminal can be electrically coupled to the electrode 327, the power supply input terminal is electrically coupled to a voltage source (e.g., a reference voltage generator), and the ground or reference terminal is electrically coupled to a ground reference electrode, which can be located on the catheter 302, can be located on a distal end of an introducer sheath, or can be located on the skin of the patient, but is not limited thereto.

In some embodiments, the ECU 402 can include a switching network configured to interchange which of electrodes of a catheter (e.g., 302) are coupled to which portions of the ECU. In some such embodiments, a user can manually switch which inputs receive connections to which electrodes of the catheter 302. Such configurability allows for a system operator to adjust the direction of propagation of the elicited potential as desired. For example, the switching network, or more generally switches, can be used to connect the electrodes 324 and 325 to the stimulator 406 during a period of time during which stimulation pulses are to be emitted by the catheter 302, and the switches can be used to connect the electrodes 326 and 326 to the amplifier 312 to sense the elicited response to the stimulation pulses. Additionally, or alternatively, a controller (e.g., 422) can autonomously control such a switching network.

The amplifier 412 can include any appropriate amplifier for amplifying desired signals or attenuating undesired signals. In some examples, the amplifier has a high common-mode rejection ratio (CMRR) for eliminating or substantially attenuating undesired signals present at each of the sensing electrodes (e.g., 326 and 327). In some embodiments, the amplifier 412 can be adjusted, for example, via an adjustable capacitance or via other attributes of the amplifier.

In the example system 400 of FIG. 4, the ECU 402 further includes a filter 414 for enhancing the desired signal in the signal received via a pair of the electrodes. The filter 414 can include a band-pass filter, a notch filter, or any other appropriate filter to isolate desired signals from noise artifacts within the received signals. In some embodiments, various properties of the filter 414 can be adjusted to manipulate its filtering characteristics. For example, the filter may include an adjustable capacitance or other parameter to adjust its frequency response.

At least one of amplification and filtering of a sensed signal (e.g., received at the electrodes 326 and 327) can allow for extraction of the desired signal at 416. In some embodiments, extraction 416 comprises at least one additional processing step to isolate desired signals from the signal sensed using electrodes such as preparing the signal for output at 418. In some embodiments, the functionalities of any combination of amplifier 412, filter 414, and extraction 416 may be combined into a single entity. For instance, the amplifier 412 may act to filter undesired frequency content from the signal without requiring additional filtering at a separate filter.

In some embodiments, the ECU 402 can record emitted stimuli and/or received signals. Such data can be subsequently stored in permanent or temporary memory 420. The ECU 402 can comprise such memory 420 or can otherwise be in communication with external memory (not shown). Thus, the ECU 402 can be configured to emit stimulus pulses to electrodes of the catheter, record such pulses in a memory, receive signals from the catheter, and also record such received signal data. The memory 420 in or associated with the ECU 402 can be internal or external to any part of the ECU 402 or the ECU 402 itself. In certain embodiments, the memory 420 stored information about evoked responses so that different evoked response can be compared to one another and decisions made in response to results of such comparisons.

The ECU 402 or separate external processor can further perform calculations on the stored data to determine characteristics of signals either emitted or received via the catheter. For example, in various embodiments, the ECU 402 can determine any of the amplitude, duration, or timing of occurrence of the received or emitted signals. The ECU 402 can further determine the relationship between the received signal and the emitted stimulus signal, such as a temporal relationship therebetween. In some embodiments, the ECU 402 performs signal averaging on the signal data received from the catheter. Such averaging can act to reduce random temporal noise in the data while strengthening the data corresponding to any elicited potentials received by the catheter.

Averaging as such can result in a signal in which temporally random noise is generally averaged out and the signal present in each recorded data set, such as elicited potentials, will remain high. In some embodiments, each iteration of the process can include a synchronization step so that each acquired data set can be temporally registered to facilitate averaging the data. That is, events that occur consistently at the same time during each iteration may be detected, while temporally random artifacts (e.g., noise) can be reduced. In general, the signal to noise ratio (SNR) resulting in such averaging will improve by the square root of the number of samples averaged in order to create the averaged data set.

The ECU 402 can further present information regarding any or all of the applied stimulus, the signal, and the results of any calculations to a user of the system, e.g., via output 418. For example, the ECU 402 can generate a graphical display providing one or more graphs of signal strength vs. time representing the stimulus and/or the received signal.

In some embodiments, the ECU 402 can include a controller 422 in communication with one or both of stimulator 406 and SENS subsystem 404. The controller 422 can be configured to cause stimulator 406 to apply a stimulation signal to a catheter, e.g., the catheter 302. Additionally or alternatively, the controller 422 can be configured to analyze signals received and/or output by the SENS subsystem 404. In some embodiments, the controller 422 can act to control the timing of applying the stimulation signal from stimulator 406 and the timing of receiving signals by the SENS subsystem 404. The controller 422 can be implemented, e.g., using one or more processors, field programmable gate arrays (FPGAs), state machines, and/or application specific integrated circuits (ASICs), but is not limited thereto.

Example electrical control units have been described. In various embodiments, the ECU 402 can emit stimulus pulses to the catheter 302, receive signals from the catheter 302, perform calculations on the emitted and/or received signals, and present the signals and/or results of such calculations to a user. In some embodiments, the ECU 402 can comprise separate modules for emitting, receiving, calculating, and providing results of calculations. Additionally or alternatively, the functionality of controller 422 can be integrated into the ECU 402 as shown, or can be separate from and in communication with the ECU.

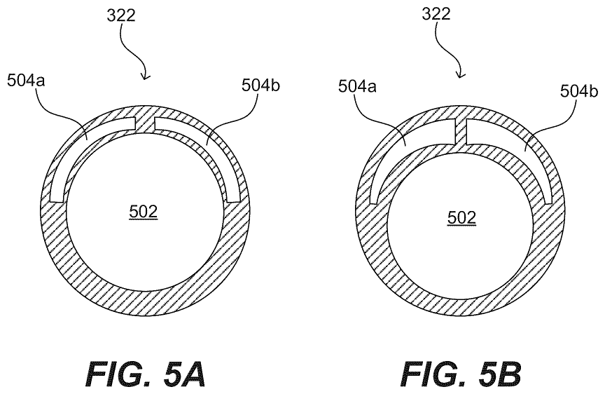

The controller 422 can also control a fluid supply subsystem 428, which can include a cartridge and a reservoir, which are described below with reference to FIG. 6, but can include alternative types of fluid pumps, and/or the like. The fluid supply subsystem 428 is fluidically coupled to one or more fluid lumens (e.g., 504a, 504b, in FIGS. 5A, 5B) within the catheter shaft 322 which in turn are fluidically coupled to the balloon 313. The fluid supply subsystem 428 can be configured to circulate a cooling liquid through the catheter 302 to the transducer 311 in the balloon 313.

The ECU 402 is also shown as including an excitation source 426 that can be used excite the transducer 311 under the control of the controller 422. For an example, the excitation source may be electrically coupled to electrodes of the transducer 311, and may actuate or excite the transducer 311 by applying a voltage between the electrodes, so as to cause the transducer 311 to emit energy. The excitation source 426, under the control of the controller 422, may be able to control the amount of energy emitted by the transducer 311 by controlling the duration of the emitted energy and/or the power of the emitted energy. The excitation source 426 can include, e.g., a voltage source, and may control the voltage and/or current output therefrom. Other variations are also possible and within the scope of the embodiments described herein.

### Example Cross-Section of Portion of Shaft of Catheter

Example cross-sections of a portion of the shaft 322 is shown in FIGS. 5A and 5B. Referring to FIG. 5A, the cross-section is shown as including a main lumen 502 having a circular cross-section, and smaller lumens 504a, 504b. In order to enable the fluid to be circulated through the balloon 313, the lumen 504a is fluidically coupled to the fluidic inlet port 334a (shown in FIG. 3) to enable fluid (e.g., expelled from a pressure syringe) to be provided to and at least partially fill the balloon 313, and the lumen 504b is fluidically coupled to the fluidic outlet port 334b (shown in FIG. 3) to enable fluid to be drawn from the balloon 313 (e.g., using a vacuum syringe). FIG. 5B shows alternative cross-sections for the lumens 502, 504b, and 504c. The main lumen 502 can function as a guide wire lumen, or the main lumen can be subdivided into additional lumen, one of which can be a guide wire lumen, and another of which can be a cable lumen that is used to hold electrical cabling that is electrically coupled to a transducer (e.g., 311) or other nerve destructive means. Other variations are also possible and within the scope of the embodiments described herein.

### Example Fluid Supply Subsystem

Example details of the fluid supply subsystem 428, which were introduced above in the discussion of FIG. 4, will now be described with reference to FIG. 6. Referring to FIG. 6, the fluid supply subsystem 428 is shown as including a cartridge 630 and a reservoir 610. The reservoir 610 is shown as being implemented as a fluid bag, which can be the same or similar to an intravenous (IV) bag in that it can hang from a hook, or the like. The reservoir 610 and the cartridge 630 can be disposable and replaceable items.

The reservoir 610 is fluidically coupled to the cartridge 630 via a pair of fluidic paths, one of which is used as a fluid outlet path (that provides fluid from the reservoir to the cartridge), and the other one of which is used as a fluid inlet path (the returns fluid from the cartridge to the reservoir). The cartridge 630 is shown as including a syringe pump 640, which includes a pressure syringe 642a and a vacuum syringe 642b. The pressure syringe 642a includes a barrel 644a, a plunger 646a, and a hub 648a. Similarly, the vacuum syringe 642b includes a barrel 644b, a plunger 646b, and a hub 648b. The hub 648a, 648b of each of the syringes 642a, 642b is coupled to a respective fluid tube or hose. The cartridge 630 is also shown as including pinch valves V1, V2 and V3, pressure sensors P1, P2, and P3, and a check valve CV. While not specifically shown in FIG. 6, the syringe pump 640 can include one or more gears and step-motors, and/or the like, which are controlled by the controller 422 (in FIG. 4) to selectively maneuver the plungers 646 of the pressure syringe 642a and the vacuum syringe 642b. Alternatively, the gear(s) and/or step-motor(s) can be used to control the syringe pump 640.

In order to at least partially fill the barrel of the pressure syringe 642a with a portion of the fluid that is stored in the reservoir 610, the pinch valves V1 and V2 are closed, the pinch valve V3 is opened, and the plunger 646a of the pressure syringe 642a is pulled upon to draw fluid 613 into the barrel 644a of the of the pressure syringe 642a. The pinch valve V3 is then closed and the pinch valves V1 and V2 are opened, and then the plunger 646a of the pressure syringe 642a is pushed upon to expel fluid from the barrel 644a of the pressure syringe 642a through the fluid tube attached to the hub 648a of the pressure syringe 642a. The fluid expelled from the pressure syringe 642a enters a fluid lumen (e.g., 504a in the catheter shaft 322), via the fluidic inlet port 334a of the catheter 302, and then enters and at least partially fills the balloon 313. Simultaneously, the plunger 646b of the vacuum syringe 642b can be pulled upon to pull or draw fluid from the balloon 313 into a fluid lumen (e.g., 504b in the catheter shaft 322), through the fluidic outlet port 334b of the catheter 302, and then through fluid tube attached to the hub 648b of the vacuum syringe 642b and into the barrel 644b of the vacuum syringe 642b. In this manner, the fluid can be circulated through the balloon 313. The balloon 313 can be inflated by supplying more fluid to the balloon than is removed from the balloon. One or more of the pressure sensors P1, P2, and P3 can be used to monitor the pressure in the balloon 313 to achieve a target balloon pressure, e.g., of 70 pounds per square inch (psi), but not limited thereto. Once the balloon 313 is inflated to a target pressure, e.g., 70 psi, and/or size, the fluid can be circulated through the balloon 313 without increasing or decreasing the amount of fluid within the balloon by causing the same amount of fluid that is removed from the balloon 313 to be the same as the amount of fluid that is provided to the balloon 313. Also, once the target balloon pressure is reached, the ultrasound transducer 311 can be excited to emit ultrasound energy to treat tissue that surrounds the portion of the biological lumen (e.g., a portion of a renal artery) in which the balloon 313 and the transducer 311 are inserted. When the ultrasound transducer 311 is emitting ultrasound energy it can also be said that the ultrasound transducer 311 is performing sonication, or that sonication is occurring. During the sonication, cooling fluid should be circulated through the balloon 313 by continuing to push on the plunger 646a of the pressure syringe 642a and continuing to pull on the plunger 646b of the vacuum syringe 642b.

As was explained above in the discussion of FIGS. 1A and 1B, in accordance with certain embodiments of the present technology, the flow rate of the cooling fluid that is circulated through the balloon 313 is one example of a denervation parameter that can be selected and used to perform a denervation parameter. Still referring to FIG. 6, the fluid supply subsystem 428 can include a cooling coil 650 within or adjacent to the reservoir 610 to control the temperature of the coiling fluid 613. As was explained above in the discussion of FIGS. 1A and 1B, in accordance with certain embodiments of the present technology, the temperature of the cooling fluid that is circulated through the balloon is another example of a denervation parameter that can be selected and used to perform a denervation procedure. The controller 422 introduced in FIG. 4 can be used to control the flow rate of the cooling fluid that is circulated through the balloon 313 by controlling the syringe pump 640. Alternatively, or additionally, the controller 422 can control the temperature of the cooling fluid 613 by controlling the temperature of the cooling coil 650.

After the sonication is completed, and the balloon 313 is to be deflated so that the catheter 302 can be removed from the biological lumen, the cooling fluid should be returned from the barrel 644b of the vacuum syringe 642b to the reservoir 610. In order to return the cooling fluid from the barrel 644b of the vacuum syringe 642b to the reservoir 610, the pinch valves V1, V2, and V3 are all closed, and the plunger of the vacuum syringe 642b is pushed on to expel the cooling fluid out of the barrel of the vacuum syringe 642b, past the check valve CV, and into the reservoir 610.

The pressure sensors P1, P2, and P3 can be used to monitor the fluidic pressure at various points along the various fluidic paths within the cartridge 630, which pressure measurements can be provided to the controller 422 as feedback that is used for controlling the syringe pump 640 and/or for other purposes, such as, but not limited to, determining the fluidic pressure within the balloon 313. Additionally, flow rate sensors F1 and F2 can be used, respectively, to monitor the flow rate of the fluid that is being injected (aka pushed, provided, or supplied) into the balloon 313, and to monitor the flow rate of the fluid that is being drawn (aka pulled or removed) from the balloon 313. The pressure measurements obtained from the pressure sensors P1, P2, and P3 can be provided to the controller 422 so that the controller 422 can monitor the balloon pressure. Additionally, flow rate measurements obtained from the flow rate sensors F1 and F2 can be provided to the controller 422 so that the controller 422 can monitor the flow rate of fluid being pushed into and pulled from the balloon 313. It would also be possible for one or more pressure sensors and/or flow rate sensors to be located at additional or alternative locations along the fluidic paths that provide fluid to and from the balloon 313.

### Example Transducer

FIGS. 7A and 7B illustrate, respectively, a longitudinal cross-sectional view and a radial cross-sectional view of an example transducer 311 of the catheter 302 introduced above in the discussion of FIGS. 3A and 3B. The transducer 311, which in the embodiment show in FIGS. 7A and 7B is an ultrasound transducer, includes a piezoelectric transducer body 701 that comprises a hollow tube of piezoelectric material having an inner surface and an outer surface, with an inner electrode 702 disposed on the inner surface of the hollow tube of piezoelectric material, and an outer electrode 703 disposed on the outer surface of the hollow tube of piezoelectric material. The hollow tube of piezoelectric material, or more generally the piezoelectric transducer body 701, is cylindrically shaped and has a circular radial cross-section. However, in alternative embodiments the transducer body 701 can have other shapes besides being cylindrical with a circular radial cross-section. The inner electrode 702 is covered by an electrical insulator 704, and the outer electrode 703 is covered by an electrical insulator 705. It is also possible that only one of the electrodes 702, 703 is covered by an electrical insulator. Other variations are also possible and within the scope of the embodiments described herein.

### Example Methods and Systems

Example 1. A method for use with a catheter including one or more electrodes and also including a transducer that can be controlled to emit a selective amount of energy, wherein the method is for use while at least a distal portion of the catheter is inserted into a biological lumen that is surrounded by tissue including nerves that are to be denervated using the transducer of the catheter, the method comprising: (a) energizing the transducer of the catheter to emit a first instance of a first amount of energy that is sufficient to heat the nerves in the tissue surrounding the biological lumen to an extent that a neural response is evoked without denervating the nerves; (b) using at least one of the one or more electrodes of the catheter to sense the neural response that is evoked by energizing the transducer to emit the first instance of the first amount of energy, and storing first information about the sensed neural response that is evoked by energizing the transducer to emit the first instance of the first amount of energy; (c) energizing the transducer of the catheter to emit a first instance of a second amount of energy that is sufficient to denervate at least some the nerves in the tissue surrounding the biological lumen; (d) after energizing the transducer of the catheter to emit the first instance of the second amount of energy, energizing the transducer of the catheter to emit a second instance of the first amount of energy; (e) using at least one of the one or more electrodes of the catheter to sense the neural response that is evoked by energizing the transducer to emit the second instance of the first amount of energy, and storing second information about the sensed neural response that is evoked by energizing the transducer to emit the second instance of the first amount of energy; and (f) comparing the second information to the first information and using results of the comparing to determine to what extent the nerves in the tissue surrounding the biological lumen were sufficiently denervated in response to the emission of the first instance of the second amount of energy.

Example 2. The method of example 1, wherein steps (a), (b), (c), (d), and (e) are performed while the transducer of the catheter is positioned at a same location within the biological lumen.

Example 3. The method of example 1, further comprising: (g) in response to determining that the nerves in the tissue surrounding the biological lumen were not sufficiently denervated in response to the emission of the first instance of the second amount of energy, energizing the transducer of the catheter to emit a second instance of the second amount of energy or energizing the transducer of the catheter to emit a third amount of energy that is greater than the second amount of energy.

Example 4. The method of example 1, wherein: the first amount of energy emitted from the transducer, that is sufficient to heat the nerves in the tissue surrounding the biological lumen to the extent that a neural response is evoked without denervating the nerves, is provided by causing the transducer to emit energy within a specified frequency range for a first duration and having a first power level that are collectively sufficient to heat the tissue surrounding the biological lumen to a temperature of at least 38 degrees Celsius, but not to exceed 52 degrees Celsius; the second amount of energy emitted from the transducer, that is sufficient to denervate at least some the nerves in the tissue surrounding the biological, is provided by causing the transducer to emit energy within the same specified frequency range for a second duration and having a second power level that are collectively sufficient to heat the tissue surrounding the biological lumen to a temperature of at least 43 degrees Celsius, but not to exceed 100 degrees Celsius; and the second duration is greater than the first duration and/or the second power level is greater than the first power level.

Example 5. The method of example 4, wherein: the second duration is greater than the first duration; and the second power level is greater than the first power level.

Example 6. The method of example 4, wherein: the second duration is greater than the first duration; and the second power level is the same as the first power level.

Example 7. The method of example 4, wherein: the second duration is the same as the first duration; and the second power level is greater than the first power level.

Example 8. The method of example 4, wherein: the transducer of the catheter comprises an ultrasound transducer; and the specified frequency range comprises 5 MHz to 20 MHz.

Example 9. The method of example 4, wherein: the transducer of the catheter comprises a radio frequency (RF) transducer; and the specified frequency range comprises 300 kHz to 600 kHz.

Example 10. The method of example 4, wherein: the transducer of the catheter comprises a microwave transducer; and the specified frequency range comprises 300 MHz to 30 GHz.

Example 11. The method of example 1, wherein the (b) using at least one of the one or more electrodes of the catheter to sense the neural response that is evoked by energizing the transducer to emit the first instance of the first amount of energy, comprises one of the following: using a pair of electrodes of the catheter to sense the neural response; using an electrode of the catheter and an electrode of a guidewire to sense the neural response; using an electrode of the catheter and an electrode of an introducer sheath to sense the neural response; or using an electrode of the catheter and an external skin electrode to sense the neural response.

Example 12. The method of example 1, wherein: the (a) energizing the transducer of the catheter to emit the first instance of the first amount of energy, and the (b) using the at least one of the one or more electrodes of the catheter to sense the neural response that is evoked by energizing the transducer to emit the first instance of the first amount of energy, are performed contemporaneously; the (d) energizing the transducer of the catheter to emit the second instance of the first amount of energy, and the (e) using the at least one of the one or more electrodes of the catheter to sense the neural response that is evoked by energizing the transducer to emit the second instance of the first amount of energy, are performed contemporaneously; and the method further comprises using a lowpass filter or a bandpass filter to distinguish the sensed neural responses to the energy emitted by the transducer from the energy emitted by the transducer.

Example 13. The method of example 1, wherein after the (a) energizing the transducer of the catheter to emit the first instance of the first amount of energy, after the (b) using the at least one of the one or more electrodes of the catheter to sense the neural response that is evoked by energizing the transducer to emit the first instance of the first amount of energy, and prior to the (d) energizing the transducer of the catheter to emit the second instance of the first amount of energy, the method further comprises: determining one or more characteristics of the sensed neural response of the nerves within the tissue surrounding the biological lumen, the one or more characteristics indicative of one or more of a size, type, function or health of the nerves and/or indicative of proximity of the nerves relative to the at least one of the one or more electrodes of the catheter; and selecting, based on the one or more characteristics of the sensed neural response, one or more denervation parameters to be used for the (c) energizing the transducer of the catheter to emit the first instance of the second amount of energy that is sufficient to denervate at least some the nerves in the tissue surrounding the biological lumen.

Example 14. A system, comprising: a catheter including one or more electrodes and also including a transducer that can be controlled to emit a selective amount of energy, wherein the catheter is configured such that at least a distal portion of the catheter is insertable into a biological lumen that is surrounded by tissue including nerves that are to be denervated using the transducer of the catheter; memory; an excitation source configured to selectively provide energy to the transducer of the catheter; a sensing subsystem electrically coupled to at least one of the one or more electrodes of the catheter; and a controller communicatively coupled to the excitation source, the sensing subsystem, and the memory, the controller configured to: cause the excitation source to energize the transducer of the catheter to emit a first instance of a first amount of energy that is sufficient to heat the nerves in the tissue surrounding the biological lumen to an extent that a neural response is evoked without denervating the nerves; cause the sensing subsystem to sense, using at least one of the one or more electrodes of the catheter, the neural response that is evoked by energizing the transducer to emit the first instance of the first amount of energy, and store in the memory first information about the sensed neural response that is evoked by energizing the transducer to emit the first instance of the first amount of energy; cause the excitation source to energize the transducer of the catheter to emit a first instance of a second amount of energy that is sufficient to denervate at least some the nerves in the tissue surrounding the biological lumen; cause the excitation source to energize the transducer of the catheter to emit a second instance of the first amount of energy, after the first instance of the second amount of energy has been emitted; cause the sensing subsystem to sense, using at least one of the one or more electrodes of the catheter, the neural response that is evoked by energizing the transducer to emit the second instance of the first amount of energy, and store in the memory second information about the sensed neural response that is evoked by energizing the transducer to emit the second instance of the first amount of energy; and compare the second information to the first information and use results of the comparison to determine to what extent the nerves in the tissue surrounding the biological lumen were sufficiently denervated in response to the emission of the first instance of the second amount of energy.

Example 15. The system of example 14, wherein the controller is further configured to: cause the excitation source to energize the transducer of the catheter to emit a second instance of the second amount of energy, in response to the controller determining that the nerves in the tissue surrounding the biological lumen were not sufficiently denervated in response to the emission of the first instance of the second amount of energy.

Example 16. The system of example 14, wherein the controller is further configured to: cause the excitation source to energize the transducer of the catheter to emit a third amount of energy that is greater than the second amount of energy, in response to the controller determining that the nerves in the tissue surrounding the biological lumen were not sufficiently denervated in response to the emission of the first instance of the second amount of energy.

Example 17. The system of example 14, wherein: the first amount of energy emitted from the transducer, that is sufficient to heat the nerves in the tissue surrounding the biological lumen to the extent that a neural response is evoked without denervating the nerves, is provided to the transducer to cause emitting of energy within a specified frequency range for a first duration and having a first power level that are collectively sufficient to heat the tissue surrounding the biological lumen to a temperature of at least 38 degrees Celsius, but not to exceed 52 degrees Celsius; the second amount of energy emitted from the transducer, that is sufficient to denervate at least some the nerves in the tissue surrounding the biological, is provided to the transducer to cause emitting of energy within the same specified frequency range for a second duration and having a second power level that are collectively sufficient to heat the tissue surrounding the biological lumen to a temperature of at least 43 degrees Celsius, but not to exceed 100 degrees Celsius; and the second duration is greater than the first duration and/or the second power level is greater than the first power level.

Example 18. The system of example 17, wherein: the second duration is greater than the first duration; and the second power level is greater than the first power level.

Example 19. The system of example 17, wherein: the second duration is greater than the first duration; and the second power level is the same as the first power level.

Example 20. The system of example 17, wherein: the second duration is the same as the first duration; and the second power level is greater than the first power level.

Example 21. The system of example 17, wherein: the transducer of the catheter comprises an ultrasound transducer; and the specified frequency range comprises 5 MHz to 20 MHz.

Example 22. The system of example 17, wherein: the transducer of the catheter comprises a radio frequency (RF) transducer; and the specified frequency range comprises 300 kHz to 600 kHz.

Example 23. The system of example 17, wherein: the transducer of the catheter comprises a microwave transducer; and the specified frequency range comprises 300 MHz to 30 GHz.

Example 24. The system of example 14, wherein the controller is configured to cause the sensing subsystem to use at least one of the one or more electrodes of the catheter to sense the neural response that is evoked by energizing the transducer to emit the first instance of the first amount of energy, by causing the sensing subsystem to: use a pair of electrodes of the catheter to sense the neural response; use an electrode of the catheter and an electrode of a guidewire to sense the neural response; use an electrode of the catheter and an electrode of an introducer sheath to sense the neural response; or use an electrode of the catheter and an external skin electrode to sense the neural response.

Example 25. The system of example 14, wherein the controller is configured to cause the sensing subsystem to: sense the neural response that is evoked by energizing the transducer to emit the first instance of the first amount of energy, contemporaneously with controlling the excitation source to energize the transducer of the catheter to emit the first instance of the first amount of energy; and sense the neural response that is evoked by energizing the transducer to emit the second instance of the first amount of energy, contemporaneously with controlling the excitation source to energize the transducer of the catheter to emit the second instance of the first amount of energy; and wherein the system further comprises a lowpass filter or a bandpass filter configured to distinguish the sensed neural responses to the energy emitted by the transducer from the energy emitted by the transducer.

Example 26. The system of example 14, wherein the controller is further configured to: determine one or more characteristics of the sensed neural response that is evoked by energizing the transducer to emit the first instance of the first amount of energy, the one or more characteristics indicative of one or more of a size, type, function or health of the nerves and/or indicative of proximity of the nerves relative to the at least one of the one or more electrodes of the catheter; and select, based on the one or more characteristics of the sensed neural response, one or more denervation parameters to be used for energizing the transducer of the catheter to emit the first instance of the second amount of energy that is sufficient to denervate at least some the nerves in the tissue surrounding the biological lumen.

Example 27. A method for use with a catheter including one or more electrodes and also including a transducer that can be controlled to emit a selective amount of energy, wherein the method is for use while at least a distal portion of the catheter is inserted into a biological lumen that is surrounded by tissue including nerves that are to be denervated using the transducer of the catheter, the method comprising: energizing the transducer of the catheter to emit a first amount of energy that is sufficient to heat the nerves in the tissue surrounding the biological lumen to an extent that a neural response is evoked without denervating the nerves; using at least one of the one or more electrodes of the catheter to sense the neural response that is evoked by energizing the transducer to emit the first amount of energy; determining one or more characteristics of the sensed neural response of the nerves within the tissue surrounding the biological lumen, the one or more characteristics indicative of one or more of a size, type, function or health of the nerves and/or indicative of proximity of the nerves relative to the at least one of the one or more electrodes of the catheter; selecting, based on the one or more characteristics of the sensed neural response, one or more denervation parameters to be used for energizing the transducer of the catheter to emit a second amount of energy that is sufficient to denervate at least some the nerves in the tissue surrounding the biological lumen.

Example 28. The method of example 27, further comprising using the selected one or more denervation parameters for energizing the transducer of the catheter to emit the second amount of energy that is sufficient to denervate at least some the nerves in the tissue surrounding the biological lumen.

Example 29. The method of example 27, further comprising using the selected one or more denervation parameters for energizing a different transducer of the catheter, or of a different catheter, to emit the second amount of energy that is sufficient to denervate at least some the nerves in the tissue surrounding the biological lumen.

Example 30. A system, comprising: a catheter including one or more electrodes and also including a transducer that can be controlled to emit a selective amount of energy, wherein the catheter is configured such that at least a distal portion of the catheter is insertable into a biological lumen that is surrounded by tissue including nerves that are to be denervated using the transducer of the catheter; memory; an excitation source configured to selectively provide energy to the transducer of the catheter; and a sensing subsystem electrically coupled to at least one of the one or more sensing electrodes of the catheter; and a controller communicatively coupled to the excitation source, the sensing subsystem, and the memory, the controller configured to: cause the excitation source to energize the transducer of the catheter to emit a first amount of energy that is sufficient to heat the nerves in the tissue surrounding the biological lumen to an extent that a neural response is evoked without denervating the nerves; cause the sensing subsystem to sense, using at least one of the one or more electrodes of the catheter, the neural response that is evoked by energizing the transducer to emit the first amount of energy; determine one or more characteristics of the sensed neural response of the nerves within the tissue surrounding the biological lumen, the one or more characteristics indicative of one or more of a size, type, function or health of the nerves and/or indicative of proximity of the nerves relative to the at least one of the one or more electrodes of the catheter; and select, based on the one or more characteristics of the sensed neural response, one or more denervation parameters to be used for energizing the transducer of the catheter to emit a second amount of energy that is sufficient to denervate at least some the nerves in the tissue surrounding the biological lumen.

Example 31. The system of example 20, wherein the controller is also configured to use the selected one or more denervation parameters to cause the excitation source to energize the transducer of the catheter to emit the second amount of energy that is sufficient to denervate at least some the nerves in the tissue surrounding the biological lumen.

Example 32. The system of example 20, wherein the controller is also configured to use the selected one or more denervation parameters to cause the excitation source to energize a different transducer of the catheter, or of a different catheter, to emit the second amount of energy that is sufficient to denervate at least some the nerves in the tissue surrounding the biological lumen.

Example 33. A system, comprising: a catheter; one or more electrodes located on a distal portion of the catheter that is configured to inserted into a biological lumen surrounded by tissue including nerves; and a transducer also located on the distal portion of the catheter that is configured to inserted into the biological lumen surrounded by the tissue including the nerves; wherein the transducer is configured to be energized to emit a first amount of energy that is sufficient to heat the nerves in the tissue surrounding the biological lumen to an extent that a neural response is evoked without denervating the nerves; and wherein at least one of the one or more electrodes of the catheter is configured to sense the neural response that is evoked by the transducer being energized to emit the first amount of energy.

Example 34. The system of example 33, wherein the neural response that is sensed is used as a baseline neural response prior to an ablation procedure being performed.

Example 35. The system of example 33, wherein the neural response that is sensed is used as a post-ablation neural response to determine an efficacy of an ablation procedure that had been performed.

Example 36. The system of example 33, wherein the neural response that is sensed is used to select one or more denervation parameters for energizing the transducer of the catheter to emit a further amount of energy that is sufficient to denervate at least some the nerves in the tissue surrounding the biological lumen.

Example 37. The system of example 33, wherein the neural response that is sensed is used to select one or more denervation parameters for energizing a different transducer of the catheter, or of a different catheter, to emit a further amount of energy that is sufficient to denervate at least some the nerves in the tissue surrounding the biological lumen.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

While the inventions are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited.

What is claimed is:

1. A method for use with a catheter including one or more electrodes and also including a transducer that can be controlled to emit a selective amount of energy, wherein the method is for use while at least a distal portion of the catheter is inserted into a biological lumen that is surrounded by tissue including nerves that are to be denervated using the transducer of the catheter, the method comprising:
(a) energizing the transducer of the catheter to emit a first instance of a first amount of energy that is sufficient to heat the nerves in the tissue surrounding the biological lumen to an extent that a neural response is evoked without denervating the nerves;
(b) using at least one of the one or more electrodes of the catheter to sense the neural response that is evoked by energizing the transducer to emit the first instance of the first amount of energy, and storing first information about the sensed neural response that is evoked by energizing the transducer to emit the first instance of the first amount of energy;
(c) energizing the transducer of the catheter to emit a first instance of a second amount of energy that is sufficient to denervate at least some the nerves in the tissue surrounding the biological lumen;
(d) after energizing the transducer of the catheter to emit the first instance of the second amount of energy, energizing the transducer of the catheter to emit a second instance of the first amount of energy;
(e) using at least one of the one or more electrodes of the catheter to sense the neural response that is evoked by energizing the transducer to emit the second instance of the first amount of energy, and storing second information about the sensed neural response that is evoked by energizing the transducer to emit the second instance of the first amount of energy; and
(f) comparing the second information to the first information and using results of the comparing to determine to what extent the nerves in the tissue surrounding the biological lumen were sufficiently denervated in response to the emission of the first instance of the second amount of energy.

2. The method of claim 1, wherein steps (a), (b), (c), (d), and (e) are performed while the transducer of the catheter is positioned at a same location within the biological lumen.

3. The method of claim 1, further comprising:
(g) in response to determining that the nerves in the tissue surrounding the biological lumen were not sufficiently denervated in response to the emission of the first instance of the second amount of energy, energizing the transducer of the catheter to emit a second instance of the second amount of energy or energizing the transducer of the catheter to emit a third amount of energy that is greater than the second amount of energy.

4. The method of claim 1, wherein:
the first amount of energy emitted from the transducer, that is sufficient to heat the nerves in the tissue surrounding the biological lumen to the extent that a neural response is evoked without denervating the nerves, is provided by causing the transducer to emit energy within a specified frequency range for a first duration and having a first power level that are collectively sufficient to heat the tissue surrounding the biological lumen to a temperature of at least 38 degrees Celsius, but not to exceed 52 degrees Celsius;
the second amount of energy emitted from the transducer, that is sufficient to denervate at least some the nerves in the tissue surrounding the biological, is provided by causing the transducer to emit energy within the same specified frequency range for a second duration and having a second power level that are collectively sufficient to heat the tissue surrounding the biological lumen to a temperature of at least 43 degrees Celsius, but not to exceed 100 degrees Celsius; and
the second duration is greater than the first duration and/or the second power level is greater than the first power level.

5. The method of claim 4, wherein:
the second duration is greater than the first duration; and
the second power level is greater than the first power level.

6. The method of claim 4, wherein:
the second duration is greater than the first duration; and
the second power level is the same as the first power level.

7. The method of claim 4, wherein:
the second duration is the same as the first duration; and
the second power level is greater than the first power level.

8. The method of claim 4, wherein:
the transducer of the catheter comprises an ultrasound transducer; and
the specified frequency range comprises 5 MHz to 20 MHz.

9. The method of claim 4, wherein:
the transducer of the catheter comprises a radio frequency (RF) transducer; and
the specified frequency range comprises 300 kHz to 600 kHz.

10. The method of claim 4, wherein:
the transducer of the catheter comprises a microwave transducer; and
the specified frequency range comprises 300 MHz to 30 GHz.

11. The method of claim 1, wherein the (b) using at least one of the one or more electrodes of the catheter to sense the neural response that is evoked by energizing the transducer to emit the first instance of the first amount of energy, comprises one of the following:
using a pair of electrodes of the catheter to sense the neural response;
using an electrode of the catheter and an electrode of a guidewire to sense the neural response;
using an electrode of the catheter and an electrode of an introducer sheath to sense the neural response; or
using an electrode of the catheter and an external skin electrode to sense the neural response.

12. The method of claim 1, wherein:
the (a) energizing the transducer of the catheter to emit the first instance of the first amount of energy, and the (b) using the at least one of the one or more electrodes of the catheter to sense the neural response that is evoked by energizing the transducer to emit the first instance of the first amount of energy, are performed contemporaneously;
the (d) energizing the transducer of the catheter to emit the second instance of the first amount of energy, and the (e) using the at least one of the one or more electrodes of the catheter to sense the neural response that is evoked by energizing the transducer to emit the second instance of the first amount of energy, are performed contemporaneously; and
the method further comprises using a lowpass filter or a bandpass filter to distinguish the sensed neural responses to the energy emitted by the transducer from the energy emitted by the transducer.

13. The method of claim 1, wherein after the (a) energizing the transducer of the catheter to emit the first instance of the first amount of energy, after the (b) using the at least one of the one or more electrodes of the catheter to sense the neural response that is evoked by energizing the transducer to emit the first instance of the first amount of energy, and prior to the (d) energizing the transducer of the catheter to emit the second instance of the first amount of energy, the method further comprises:
determining one or more characteristics of the sensed neural response of the nerves within the tissue surrounding the biological lumen, the one or more characteristics indicative of one or more of a size, type, function or health of the nerves and/or indicative of proximity of the nerves relative to the at least one of the one or more electrodes of the catheter; and
selecting, based on the one or more characteristics of the sensed neural response, one or more denervation parameters to be used for the (c) energizing the transducer of the catheter to emit the first instance of the second amount of energy that is sufficient to denervate at least some the nerves in the tissue surrounding the biological lumen.

14. A system, comprising:
a catheter including one or more electrodes and also including a transducer that can be controlled to emit a selective amount of energy, wherein the catheter is configured such that at least a distal portion of the catheter is insertable into a biological lumen that is surrounded by tissue including nerves that are to be denervated using the transducer of the catheter;
memory;
an excitation source configured to selectively provide energy to the transducer of the catheter;
a sensing subsystem electrically coupled to at least one of the one or more electrodes of the catheter; and
a controller communicatively coupled to the excitation source, the sensing subsystem, and the memory, the controller configured to:
cause the excitation source to energize the transducer of the catheter to emit a first instance of a first amount of energy that is sufficient to heat the nerves in the tissue surrounding the biological lumen to an extent that a neural response is evoked without denervating the nerves;
cause the sensing subsystem to sense, using at least one of the one or more electrodes of the catheter, the neural response that is evoked by energizing the transducer to emit the first instance of the first amount of energy, and store in the memory first information about the sensed neural response that is evoked by energizing the transducer to emit the first instance of the first amount of energy;
cause the excitation source to energize the transducer of the catheter to emit a first instance of a second amount of energy that is sufficient to denervate at least some the nerves in the tissue surrounding the biological lumen;
cause the excitation source to energize the transducer of the catheter to emit a second instance of the first amount of energy, after the first instance of the second amount of energy has been emitted;
cause the sensing subsystem to sense, using at least one of the one or more electrodes of the catheter, the neural response that is evoked by energizing the transducer to emit the second instance of the first amount of energy, and store in the memory second information about the sensed neural response that is evoked by energizing the transducer to emit the second instance of the first amount of energy; and
compare the second information to the first information and use results of the comparison to determine to what extent the nerves in the tissue surrounding the biological lumen were sufficiently denervated in response to the emission of the first instance of the second amount of energy.

15. The system of claim 14, wherein the controller is further configured to:
cause the excitation source to energize the transducer of the catheter to emit a second instance of the second amount of energy, in response to the controller determining that the nerves in the tissue surrounding the biological lumen were not sufficiently denervated in response to the emission of the first instance of the second amount of energy.

16. The system of claim 14, wherein the controller is further configured to:
cause the excitation source to energize the transducer of the catheter to emit a third amount of energy that is greater than the second amount of energy, in response to the controller determining that the nerves in the tissue surrounding the biological lumen were not sufficiently denervated in response to the emission of the first instance of the second amount of energy.

17. The system of claim 14, wherein:
the first amount of energy emitted from the transducer, that is sufficient to heat the nerves in the tissue surrounding the biological lumen to the extent that a neural response is evoked without denervating the nerves, is provided to the transducer to cause emitting of energy within a specified frequency range for a first duration and having a first power level that are collectively sufficient to heat the tissue surrounding the biological lumen to a temperature of at least 38 degrees Celsius, but not to exceed 52 degrees Celsius;
the second amount of energy emitted from the transducer, that is sufficient to denervate at least some the nerves in the tissue surrounding the biological, is provided to the transducer to cause emitting of energy within the same specified frequency range for a second duration and having a second power level that are collectively sufficient to heat the tissue surrounding the biological lumen to a temperature of at least 43 degrees Celsius, but not to exceed 100 degrees Celsius; and
the second duration is greater than the first duration and/or the second power level is greater than the first power level.

18. The system of claim 17, wherein:
the second duration is greater than the first duration; and
the second power level is greater than the first power level.

19. The system of claim 17, wherein:
the second duration is greater than the first duration; and
the second power level is the same as the first power level.

20. The system of claim 17, wherein:
the second duration is the same as the first duration; and
the second power level is greater than the first power level.

21. The system of claim 17, wherein:
the transducer of the catheter comprises an ultrasound transducer; and
the specified frequency range comprises 5 MHz to 20 MHz.

22. The system of claim 17, wherein:
the transducer of the catheter comprises a radio frequency (RF) transducer; and
the specified frequency range comprises 300 kHz to 600 kHz.

23. The system of claim 17, wherein:
the transducer of the catheter comprises a microwave transducer; and
the specified frequency range comprises 300 MHz to 30 GHz.

24. The system of claim 14, wherein the controller is configured to cause the sensing subsystem to use at least one of the one or more electrodes of the catheter to sense the neural response that is evoked by energizing the transducer to emit the first instance of the first amount of energy, by causing the sensing subsystem to:
use a pair of electrodes of the catheter to sense the neural response;
use an electrode of the catheter and an electrode of a guidewire to sense the neural response;
use an electrode of the catheter and an electrode of an introducer sheath to sense the neural response; or
use an electrode of the catheter and an external skin electrode to sense the neural response.

25. The system of claim 14, wherein the controller is configured to cause the sensing subsystem to:
sense the neural response that is evoked by energizing the transducer to emit the first instance of the first amount of energy, contemporaneously with controlling the excitation source to energize the transducer of the catheter to emit the first instance of the first amount of energy; and
sense the neural response that is evoked by energizing the transducer to emit the second instance of the first amount of energy, contemporaneously with controlling the excitation source to energize the transducer of the catheter to emit the second instance of the first amount of energy; and
wherein the system further comprises a lowpass filter or a bandpass filter configured to distinguish the sensed neural responses to the energy emitted by the transducer from the energy emitted by the transducer.

26. The system of claim 14, wherein the controller is further configured to:
determine one or more characteristics of the sensed neural response that is evoked by energizing the transducer to emit the first instance of the first amount of energy, the one or more characteristics indicative of one or more of a size, type, function or health of the nerves and/or indicative of proximity of the nerves relative to the at least one of the one or more electrodes of the catheter; and
select, based on the one or more characteristics of the sensed neural response, one or more denervation parameters to be used for energizing the transducer of the catheter to emit the first instance of the second amount of energy that is sufficient to denervate at least some the nerves in the tissue surrounding the biological lumen.

27. A method for use with a catheter including one or more electrodes and also including a transducer that can be controlled to emit a selective amount of energy, wherein the method is for use while at least a distal portion of the catheter is inserted into a biological lumen that is surrounded by tissue including nerves that are to be denervated using the transducer of the catheter, the method comprising:
energizing the transducer of the catheter to emit a first amount of energy that is sufficient to heat the nerves in the tissue surrounding the biological lumen to an extent that a neural response is evoked without denervating the nerves;
using at least one of the one or more electrodes of the catheter to sense the neural response that is evoked by energizing the transducer to emit the first amount of energy;
determining one or more characteristics of the sensed neural response of the nerves within the tissue surrounding the biological lumen, the one or more characteristics indicative of one or more of a size, type, function or health of the nerves and/or indicative of proximity of the nerves relative to the at least one of the one or more electrodes of the catheter; and
selecting, based on the one or more characteristics of the sensed neural response, one or more denervation parameters to be used for energizing the transducer of the catheter to emit a second amount of energy that is sufficient to denervate at least some the nerves in the tissue surrounding the biological lumen.

28. The method of claim 27, further comprising using the selected one or more denervation parameters for energizing the transducer of the catheter to emit the second amount of energy that is sufficient to denervate at least some the nerves in the tissue surrounding the biological lumen.

29. The method of claim 27, further comprising using the selected one or more denervation parameters for energizing a different transducer of the catheter, or of a different catheter, to emit the second amount of energy that is sufficient to denervate at least some the nerves in the tissue surrounding the biological lumen.

30. A system, comprising:
a catheter including one or more electrodes and also including a transducer that can be controlled to emit a selective amount of energy, wherein the catheter is configured such that at least a distal portion of the catheter is insertable into a biological lumen that is surrounded by tissue including nerves that are to be denervated using the transducer of the catheter;
memory;
an excitation source configured to selectively provide energy to the transducer of the catheter; and
a sensing subsystem electrically coupled to at least one of the one or more sensing electrodes of the catheter; and
a controller communicatively coupled to the excitation source, the sensing subsystem, and the memory, the controller configured to:
cause the excitation source to energize the transducer of the catheter to emit a first amount of energy that is sufficient to heat the nerves in the tissue surrounding the biological lumen to an extent that a neural response is evoked without denervating the nerves;
cause the sensing subsystem to sense, using at least one of the one or more electrodes of the catheter, the neural response that is evoked by energizing the transducer to emit the first amount of energy;
determine one or more characteristics of the sensed neural response of the nerves within the tissue surrounding the biological lumen, the one or more characteristics indicative of one or more of a size, type, function or health of the nerves and/or indicative of proximity of the nerves relative to the at least one of the one or more electrodes of the catheter; and
select, based on the one or more characteristics of the sensed neural response, one or more denervation parameters to be used for energizing the transducer of the catheter to emit a second amount of energy that is sufficient to denervate at least some the nerves in the tissue surrounding the biological lumen.

31. The system of claim 30, wherein the controller is also configured to use the selected one or more denervation parameters to cause the excitation source to energize the transducer of the catheter to emit the second amount of energy that is sufficient to denervate at least some the nerves in the tissue surrounding the biological lumen.

32. The system of claim 30, wherein the controller is also configured to use the selected one or more denervation parameters to cause the excitation source to energize a different transducer of the catheter, or of a different catheter, to emit the second amount of energy that is sufficient to denervate at least some the nerves in the tissue surrounding the biological lumen.

33. A system, comprising:
a catheter;
one or more electrodes located on a distal portion of the catheter that is configured to inserted into a biological lumen surrounded by tissue including nerves; and
a transducer also located on the distal portion of the catheter that is configured to inserted into the biological lumen surrounded by the tissue including the nerves;
wherein the transducer is configured to be energized to emit a first amount of energy that is sufficient to heat the nerves in the tissue surrounding the biological lumen to an extent that a neural response is evoked without denervating the nerves; and
wherein at least one of the one or more electrodes of the catheter is configured to sense the neural response that is evoked by the transducer being energized to emit the first amount of energy.

34. The system of claim 33, wherein the neural response that is sensed is used as a baseline neural response prior to an ablation procedure being performed.

35. The system of claim 33, wherein the neural response that is sensed is used as a post-ablation neural response to determine an efficacy of an ablation procedure that had been performed.

36. The system of claim 33, wherein the neural response that is sensed is used to select one or more denervation parameters for energizing the transducer of the catheter to emit a further amount of energy that is sufficient to denervate at least some the nerves in the tissue surrounding the biological lumen.

37. The system of claim 33, wherein the neural response that is sensed is used to select one or more denervation parameters for energizing a different transducer of the catheter, or of a different catheter, to emit a further amount of energy that is sufficient to denervate at least some the nerves in the tissue surrounding the biological lumen.
